(19) United States
(12) Patent Application Publication
(10) Pub. No.: US 2024/0115681 A1
(43) Pub. Date: Apr. 11, 2024

(54) PHARMACEUTICAL COMPOSITION COMPRISING TOLL-LIKE RECEPTOR AGONIST AND STIMULATOR OF INTERFERON GENES AGONIST AND USE THEREOF

(71) Applicant: National Health Research Institutes, Miaoli County (TW)

(72) Inventors: Tsung-Hsien Chuang, Miaoli County (TW); Jing-Xing Yang, Miaoli County (TW); Jen-Chih Tseng, Miaoli County (TW); Zaida Nur Imana, Miaoli County (TW); Ming-Hsi Huang, Miaoli County (TW); Guann-Yi Yu, Miaoli County (TW)

(73) Assignee: National Health Research Institutes, Miaoli County (TW)

(21) Appl. No.: 18/476,345

(22) Filed: Sep. 28, 2023

Related U.S. Application Data
(60) Provisional application No. 63/377,839, filed on Sep. 30, 2022.

(51) Int. Cl. A61K 39/00; A61K 9/00; A61K 39/39; A61P 31/16; A61P 35/00

(52) U.S. Cl. CPC A61K 39/001111; A61K 9/0019; A61K 9/0043; A61K 39/39; A61P 31/16; A61P 35/00; A61K 2039/543; A61K 2039/55561

(57) ABSTRACT

Provided is a pharmaceutical composition including an active pharmaceutical ingredient, a toll-like receptor (TLR) agonist, a stimulator of interferon genes (STING) agonist, and a pharmaceutically acceptable carrier. Also provided are a method for inducing immune response and a method for treating or preventing cancer or an infectious disease, including administering an effective amount of the pharmaceutical composition to a subject in need thereof.

### CROSS REFERENCE TO RELATED APPLICATIONS

This application claims the benefit of U.S. Provisional Application No. 63/377,839, filed on Sep. 30, 2022. The content of the application is incorporated herein by reference.

### BACKGROUND OF THE INVENTION

### 1. Field of the Invention

The present disclosure relates to vaccines and immunotherapy, and particularly to an adjuvant composition for enhancing the effect thereof.

### 2. Description of the Prior Art

Infectious diseases are a major threat to human life and can lead to global health crises. As a result, the goal of immunotherapy is to activate antigen-specific immune responses or reactivate pre-existing responses against pathogenic invaders or tumor cells in the immune system. Its effect can be further enhanced by using vaccine adjuvants. The stimulator of interferon genes (STING) pathway plays an important role in innate immunity, promoting a unique immune effector response and distinguishing pathogens and host cells by detecting extracellular and intracellular danger signals. Moreover, STING is the first line of defense against viral and bacterial infections and malignant cells. However, pathogens, as well as tumor cells, have evolved ways to evade recognition by the immune system.

For example, in 2019, COVID-19 pandemic caused a global health crisis, but the COVID-19 virus has the characteristics of continuous mutation, and the vaccine protection may gradually decline with the time, so regular vaccination has become an important approach against COVID-19. The current COVID-19 vaccine is designed to induce antibodies that neutralize the entry of SARS-CoV-2 by recombinant S protein or using the receptor binding domain (RBD) as the target antigen. However, the use of recombinant antigens often shows weak immunogenicity. Therefore, the use of adjuvants to assist vaccines still requires further research.

On the other hand, in the case of tumor cell invasion, inhibition of immune checkpoints becomes a possible solution, and most immunotherapy drugs are immune checkpoint inhibitors. For example, inhibiting programmed cell death protein 1 (PD-1) and other receptors can enhance the activity of immune cells, thereby enabling immune cells to effectively identify and eliminate tumor cells in the human body.

Thus, there is still an unmet need in the art of using adjuvants to further stimulate the immune system against pathogenic invasion or tumor cells. Furthermore, there is an urgent need in this art for effective adjuvants thereby stimulating the immune system.

### SUMMARY OF THE INVENTION

The present disclosure provides a pharmaceutical composition comprising an active pharmaceutical ingredient, a toll-like receptor (TLR) agonist, a stimulator of interferon genes (STING) agonist, and a pharmaceutically acceptable carrier.

In at least one embodiment of the present disclosure, the toll-like receptor agonist is a toll-like receptor 9 (TLR 9) agonist or a toll-like receptor 21 (TLR 21) agonist. In some embodiments, the subject is a mammalian animal (e.g., a human or a murine), and the toll-like receptor agonist is TLR 9. In some other embodiments, the subject is a non-mammalian animal (e.g., a bird or a chicken), and the toll-like receptor agonist is TLR 21

In at least one embodiment of the present disclosure, the toll-like receptor agonist is a CpG-oligodeoxynucleotide.

In at least one embodiment of the present disclosure, the CpG-oligodeoxynucleotide comprises a sequence being at least 70%, 75%, 80%, 85%, 90%, 95%, 96%, 97%, 98%, or 99% identical to SEQ ID NO: 31. In some embodiment, the CpG-oligodeoxynucleotide comprises a sequence of SEQ ID NO: 31.

In at least one embodiment of the present disclosure, the stimulator of interferon genes agonist is a cyclic di-nucleotide.

In at least one embodiment of the present disclosure, the cyclic di-nucleotide is at least one selected from the group consisting of 2′3′-cGAMP, 3′3′-cGAMP, 2′3′-cGAM(PS)2, c-di-AM(PS)2, c-di-AMP, 2′2′-cGAMP, and 2′3′-c-di-AM(PS)2. In some embodiments, the cyclic di-nucleotide is at least one selected from the group consisting of 2′2′-cGAMP, 2′3′-cGAMP, cGAM(PS)2, and c-di-AM(PS)2. In some embodiments, the cyclic di-nucleotide is 2′3′-c-di-AM(PS)2 or c-di-AMP. In some embodiments, the cyclic di-nucleotide is 2′3′-c-di-AM(PS)2. In some embodiments, the cyclic di-nucleotide is c-di-AMP.

The present disclosure also provided a method for an inducing immune response in a subject in need thereof, comprising administering an effective amount of the pharmaceutical composition of the present disclosure to the subject.

In at least one embodiment of the present disclosure, the effective amount is about 0.01 mg/kg body weight to about 20 mg/kg body weight, about 0.05 mg/kg body weight to about 15 mg/kg body weight, about 0.1 mg/kg body weight to about 10 mg/kg body weight, about 0.15 mg/kg body weight to about 5 mg/kg body weight, about 0.2 mg/kg body weight to about 1 mg/kg body weight, about 0.25 mg/kg body weight to about 1 mg/kg body weight, about 0.3 mg/kg body weight to about 1 mg/kg body weight, about 0.35 mg/kg body weight to about 1 mg/kg body weight, about 0.4 mg/kg body weight to about 1 mg/kg body weight, about 0.45 mg/kg body weight to about 1 mg/kg body weight, about 0.5 mg/kg body weight to about 1 mg/kg body weight, about 0.55 mg/kg body weight to about 1 mg/kg body weight, about 0.6 mg/kg body weight to about 1 mg/kg body weight, about 0.65 mg/kg body weight to about 1 mg/kg body weight, about 0.7 mg/kg body weight to about 1 mg/kg body weight, about 0.75 mg/kg body weight to about 1 mg/kg body weight, about 0.8 mg/kg body weight to about 1 mg/kg body weight, about 0.85 mg/kg body weight to about 1 mg/kg body weight, about 0.9 mg/kg body weight to about 1 mg/kg body weight, and about 0.95 mg/kg body weight to about 1 mg/kg body weight.

In at least one embodiment of the present disclosure, pharmaceutical composition is administered to the subject by intramuscular injection, subcutaneous administration, nasal administration or intratumoral administration.

The present disclosure also provided a method treating or preventing cancer or an infectious disease, comprising administering an effective amount of the pharmaceutical composition of the present disclosure to a subject in need thereof.

In at least one embodiment of the present disclosure, the active pharmaceutical ingredient is an immune check point inhibitor. In some embodiments, the immune check point inhibitor inhibits programmed cell death protein 1 receptor, programmed cell death ligand 1 receptor, and cytotoxic T-lymphocyte-associated antigen 4 receptor.

In at least one embodiment of the present disclosure, the cancer is at least one selected from the group consisting of head and neck cancer, breast cancer, prostate cancer, melanoma, lymphoma, non-small-cell lung cancer, basal cell carcinoma, glioblastoma, and ovarian cancer.

In at least one embodiment of the present disclosure, the infectious disease is induced by hepatitis B virus, anthrax, malaria, pneumonia, herpes simplex virus, influenza virus, or any combination thereof.

Also provided herein is a use of the pharmaceutical composition of the present disclosure mentioned above for manufacture of a medicament for treating or preventing cancer or an infectious disease in a subject in need thereof.

In at least one embodiment of the present disclosure, adjuvants formulated with a Toll-like receptor (TLR) agonist, CpG-2722, with various cyclic dinucleotides (CDNs) that are STING agonists increased germinal center B cell response and elicited humoral immune responses.

These and other objectives of the present invention will no doubt become obvious to those of ordinary skill in the art after reading the following detailed description of the preferred embodiment that is illustrated in the various figures and drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A, FIG. 1B, FIG. 1C, and FIG. 1D show RBD protein adjuvanted with combined CpG-2722/STING agonists induced a robust humoral response after intramuscular immunization. FIG. 1A shows the schematic illustration of the experimental design. BALB/c mice were immunized intramuscularly on days 0, 11, and 21 with a vaccine formulated with 10 μg RBD protein, 10 μg CpG-2722, and 5 μg of different cyclic-dinucleotides (CDNs) as indicated. FIG. 1B shows serum samples collected on days 10, 20, and 30, and the total amount of anti-S protein IgG thereof was quantified with ELISA. Serum samples collected on day 30 were subjected to the hACE2-RBD competition assay (shown in FIG. 1C) and the SARS-CoV-2 neutralization assay (shown in FIG. 1D). Data are the mean±SEM (n=5/group). *P<0.05, **P<0.01, and ***P<0.001 compared with the group of mice treated with PBS vehicle control or between the indicated groups. CpG, cGAM(PS)2, and c-di-AM(PS)2 stand for CpG-2722, 2′3′-cGAM(PS)2, and 2′3′-c-di-AM(PS)2, respectively. + stands for plus RBD protein antigen.

FIG. 2A, FIG. 2B, FIG. 2C, FIG. 2D, FIG. 2D, FIG. 2E, FIG. 2F, and FIG. 2G show RBD adjuvanted with CpG-2722/c-di-AM(PS)2 induced a robust humoral response via intranasal inoculation. FIG. 2A shows the schematic illustration of the experimental design. BALB/c mice were immunized intranasally on days 0, 11, and 21 with a vaccine formulated with 10 μg of RBD protein, 10 μg CpG-2722, and 5 μg 2′3′c-di-AM(PS)2. Serum samples were collected on days 10, 20, and 30. The bronchoalveolar lavage fluid (BALF), nasal lavage fluid (NLF), and nasal tissue of the vaccinated mice were collected 10 days after vaccination (day 30). The total amount of anti-S IgG and IgA from serum (shown in FIG. 2B), BALF, and NLF (shown in FIG. 2C) were quantified with ELISA. Serum samples collected on day 30 were subjected to the hACE2-RBD competition assay (shown in FIG. 2D), an S protein-containing VSV pseudovirus neutralization assay (shown in FIG. 2E), and the SARS-CoV-2 neutralization assay (shown in FIG. 2F). Representative H&E stained nasal tissues at 10 days post-challenge are shown in FIG. 2G. Data are the mean±SEM (n=5/group). ***P<0.001 compared with mice treated with PBS vehicle control or between the different groups as indicated. CpG and AM(PS)2 stand for CpG-2722 and 2′3′-c-di-AM(PS)2. + stands for plus RBD protein antigen.

FIG. 3A, FIG. 3B, FIG. 3C, and FIG. 3D show a combination of CpG-2722 and STING ligands elicited germinal center (GC) T follicular helper (Tfh) cell and B cell responses. In the experiment of FIG. 1, the mice were euthanized 10 days after the final vaccination (day 30) for the collection of draining lymph nodes and spleens. The numbers of Tfh cells (CD4+Bcl-6+CXCR5+ICOS+) and GC B cells (B220+CO19+CD38−GL7+FAS+) from lymph nodes (shown in FIG. 3A and FIG. 3B) and spleens (shown in FIG. 3C and FIG. 3D) were measured by flow cytometry. Data are the mean±SEM (n=5/group). *P<0.05, **P<0.01, and ***P<0.001 between the different groups as indicated. CpG, GAM(PS)2, and AM(PS)2 stand for CpG-2722, 2′3′-cGAM(PS)2 and 2′3′-c-di-AM(PS)2. + stands for plus RBD protein antigen.

FIG. 4A, FIG. 4B, and FIG. 4C show cooperative adjuvant effect of CpG-2722 and c-di-AM(PS)2 on inducing a humoral response to the RBD protein vaccine. BALB/c mice were immunized intramuscularly on days 0, 11, and 21 with 10 μg of the RBD protein vaccine adjuvanted with 10 μg CpG-2722 and 5 μg c-di-AM(PS)2 alone or in combination. Serum samples were collected on days 30. As shown in FIG. 4A, the total amount of anti-S protein IgG (left panel) and IgA (right panel) were quantified with ELISA. As shown in FIG. 4B, these samples were subjected to the S-containing VSV pseudovirus neutralization assay. As shown in FIG. 4C, the SARS-CoV-2 neutralization assay. Data are the mean±SEM (n=5/group). *P<0.05, **P<0.01, and ***P<0.001 compared with mice treated with PBS vehicle control or between the different groups as indicated. CpG and AM(PS)2 stand for CpG-2722 and 2′3′-c-di-AM(PS)2. + stands for plus RBD protein antigen.

FIG. 5A, FIG. 5B, and FIG. 5C show antigen-dependent T helper responses in mice immunized with RBD protein vaccines adjuvanted with CpG-2722 and c-di-AM(PS)2 alone or in combination. In the experiments of FIG. 4, mice were euthanized 10 days after the final immunization. As shown in FIG. 5A, FIG. 5B, and FIG. 5C, splenocytes were prepared and stimulated with RBD for 96 h. The concentrations of signature cytokines for Th1 (IFN-γ) (shown in FIG. 5A), Th17 (IL-17A) (shown in FIG. 5B), and Th2 (IL-4, IL-5, IL-13) (shown in FIG. 5C) in the culture supernatants were measured by ELISA. Data are the mean±SEM (n=5/group). *P <0.05, **P<0.01, and ***P<0.001 compared with mice treated with PBS vehicle control or between the different groups as indicated. CpG and AM(PS)2 stand for CpG-2722 and 2′3′-c-di-AM(PS)2. + stands for plus RBD protein antigen.

FIG. 6A and FIG. 6B show CpG-2722 and c-di-AM(PS)2 cooperatively promoted the activation of bone marrow derived macrophages (BMDCs). Mouse BMDCs were stimulated with CpG-2722 (5 μg/ml), c-di-AM(PS)2 (1 μg/ml), or a combination of both. After 24 h, cells were collected and analyzed the surface expression of CD11c, CD40, CD80, CD86, and CCR7 by flow cytometry. FIG. 6A, shows a representative histograms. As shown in FIG. 6B, the MFI of CD40+, CD80+, CD86+, and CCR7+ cells in a population of CD11c+ cells were quantified. Data are the mean±SEM (n=4). *P<0.05, **P<0.01, and ***P<0.001 compared with control cells or between the different groups as indicated. CpG and AM(PS)2 stand for CpG-2722 and 2′3′-c-di-AM(PS)2.

FIG. 7 shows effect of CpG-2722, c-di-AM(PS)2, and their combination on cytokine expression in mouse BMDCs. Mouse BMDCs were cultured with CpG-2722 (5 μg/ml), c-di-AM(PS)2 (1 μg/ml), or a combination of both. After 4 h, the total RNA was isolated. The expression of different cytokines in the cells was determined by RT-qPCR and normalized to β-actin levels. Data are presented as fold induction compared with the mRNA level in PBS vehicle control treated cells. Data are the mean±SEM (n=4). *P<0.05, **P<0.01, and ***P<0.001 compared with control cells or between the different groups as indicated. CpG and AM(PS)2 stand for CpG-2722 and 2′3′-c-di-AM(PS)2.

FIG. 8 shows effect of CpG-2722, c-di-AM(PS)2, and their combination on cytokine expression in mouse splenocytes. Mouse splenocytes were cultured with CpG-2722 (5 μg/ml), c-di-AM(PS)2 (1 μg/ml), or a combination of both. After 4 h, the total RNA was isolated. The expression of different cytokines in the cells was determined by RT-qPCR and normalized to β-actin levels. Data are presented as fold induction compared with the mRNA level in PBS vehicle control treated cells. Data are the mean±SEM (n=3). *P<0.05, **P<0.01, and ***P<0.001 compared with control cells or between the different groups as indicated. CpG and AM(PS)2 stand for CpG-2722 and 2′3′-c-di-AM(PS)2.

FIG. 9 shows effect of CpG-2722, c-di-AM(PS)2, and their combination on cytokine expression in mouse peripheral blood mononuclear cells (PBMCs). Mouse PBMCs were cultured with CpG-2722 (5 μg/ml), c-di-AM(PS)2 (1 μg/ml), or a combination of both. After 4 h, the total RNA was isolated. The expression of different cytokines in the cells was determined by RT-qPCR and normalized to β-actin levels. Data are presented as fold induction compared with the mRNA level in PBS vehicle control treated cells. Data are the mean±SEM (n=3). *P<0.05, **P<0.01, and ***P<0.001 compared with control cells or between the different groups as indicated. CpG and AM(PS)2 stand for CpG-2722 and 2′3′-c-di-AM(PS)2.

FIG. 10 shows effect of CpG-2722 and c-di-AM(PS)2 on cytokine production in mouse embryonic fibroblasts (MEFs). MEFs were cultured with CpG-2722 (5 μg/ml), c-di-AM(PS)2 (1 μg/ml), or a combination of both. After 4 h, the total RNA was isolated. The expression of different cytokines in the cells was determined by RT-qPCR and normalized to β-actin levels. Data are presented as fold induction compared with the mRNA level in PBS vehicle control treated cells. Data are the mean±SEM (n=3). *P<0.05, **P<0.01, and ***P<0.001 compared with control cells or between the different groups as indicated. CpG and AM(PS)2 stand for CpG-2722 and 2′3′-c-di-AM(PS)2.

FIG. 11 shows a schematic diagram cooperative illustrating adjuvant activity of the TLR9 agonist and STING agonist combination. The TLR9 agonist, CpG-2722, and the STING agonist, c-di-AM(PS)2, cooperatively boost the immune response to SARS-CoV-2 RBD vaccine through an increased germinal center B cell response and reshaped T helper responses.

FIG. 12A shows structural features of CpG-2722. FIG. 12B show structural features of 2′2′-cGAMP, 2′3′-cGAMP, 2′3′-cGAM(PS)2, and 2′3′-cdi-AM(PS)2 and the comparison thereof.

FIG. 13A and FIG. 13B show the combination of CpG-2722 and STING ligands elicited robust Tfh cell responses. *P<0.05, **P<0.01, ***P<0.001, compared with mice treated with PBS vehicle control or between the different groups as indicated. CpG and AM(PS)2 stand for CpG-2722 and 2′3′-c-di-AM(PS)2. + stands for plus RBD protein antigen.

FIG. 14A, FIG. 14B, and FIG. 14C show cooperative adjuvant effect of CpG-2722 and c-di-AM(PS)2 on inducing a humoral response to the RBD protein vaccine. *P<0.05, **P<0.01, and ***P<0.001 compared with mice treated with PBS vehicle control or between the different groups as indicated. CpG and AM(PS)2 stand for CpG-2722 and 2′3′-c-di-AM(PS)2. + stands for plus RBD protein antigen.

FIG. 15A, FIG. 15B, and FIG. 15C show adjuvant activities of CpG-2722 and 2′3′-c-di-AMP or 2′3′-c-di-AM(PS)2 combinations for intranasally administrated influenza viral like particle vaccines. *P<0.05, **P<0.01, and ***P<0.001 compared with mice treated with PBS vehicle control or between the different groups as indicated. VLP stands for viral like particle.

FIG. 16A and FIG. 16B show cooperative effect of CpG-2722 and 2′3′-c-di-AMP on the suppression of head and neck tumor growth. *P<0.05, **P<0.01, and ***P<0.001 compared with mice treated with PBS vehicle control or between the different groups as indicated.

### DETAILED DESCRIPTION

The following embodiments are provided to illustrate the present disclosure in detail. A person having ordinary skill in the art can easily understand the advantages and effects of the present disclosure after reading the disclosure of this specification, and also can implement or apply in other different embodiments. Therefore, it is possible to modify and/or alter the following embodiments for carrying out this disclosure without contravening its scope for different aspects and applications, and any element or method within the scope of the present disclosure disclosed herein can combine with any other element or method disclosed in any embodiments of the present disclosure.

The articles “a”, “an”, and “the” are used herein to refer to one or to more than one (i.e., to at least one) of the grammatical object of the article. The term “or” is used interchangeably with the term “and/or” unless the context clearly indicates otherwise. The term “including” is used herein to mean, and is used interchangeably with, the phrase “including but not limited to.” The term “about” is used herein to mean within the typical ranges of tolerances in the art. For example, “about” can be understood as about 2 standard deviations from the mean. When about is present before a series of numbers or a range, it is understood that “about” can modify each of the numbers in the series or range. For example, the numerical value is meant to encompass variations of ±20%, ±10%, ±5%, ±1%, ±0.5%, or ±0.1% from the numerical value. The numeral ranges used herein are inclusive and combinable, any numeral value that falls within the numeral scope herein could be taken as a maximum or minimum value to derive the sub-ranges therefrom. For example, it should be understood that the numeral range “20-30%” comprises any sub-ranges between the minimum value of 20% to the maximum value of 30%, such as the sub-ranges from 20% to 25%, from 25% to 30%, and from 22.5% to 27.5%. Such variations in the numerical value may occur by, e.g., the experimental error, the typical error in measuring or handling procedure for making compounds, compositions, concentrates, or formulations, the differences in the source, manufacture, or purity of starting materials or ingredients used in the present disclosure, or like considerations.

As used herein, the terms “effective amount,” refers to the amount of an active agent or a pharmaceutical composition that is sufficient to bring about a therapeutic effect on a subject in need thereof. The effective amount may vary by a person ordinarily skilled in the art, depending on excipient usage, routes of administration, the possibility of co-usage with other therapeutic treatment, or the condition to be treated, but the present disclosure is not limited thereto.

As used herein, the term “administer,” “administering” or “administration” refers to the placement of an active ingredient into a subject by a method or route which results in at least partial localization of the active ingredient at a desired site to produce the desired effect. For example, the active ingredient of the present disclosure may be administered to the subject by injection, subcutaneous administration, or nasal administration, but the present disclosure is not limited thereto.

As used here, the term “subject” as used herein includes both mammalian and non-mammalian animals. The mammalian animals include, but not limited to, humans, non-human primates, canines, felines, murines, bovines, equines, porcines, sheeps, deers, wolfs, foxes, and rabbits. The non-mammalian animals include, but not limited to, class Ayes (e.g., birds or chickens) and fishes.

The terms “comprise,” “comprising,” “include,” “including,” “have,” “having,” “contain,” “containing,” and any other variations thereof are intended used herein to cover a non-exclusive inclusion. For example, when describing an object “comprises” a limitation, unless otherwise specified, it may additionally include other ingredients, elements, components, structures, regions, parts, devices, systems, steps, or connections, etc., and should not exclude other limitations.

As used herein, the term “pharmaceutically acceptable carrier” refers to a pharmaceutically acceptable material, vehicle, or composition, such as a solid or liquid filler, binder, diluent, preservative, biocompatible solvent, disintegrating agent, lubricant, suspending agent, flavoring agent, encapsulating material, thickening agent, acid, surfactant, complexation agent, wetting agent, or any combination thereof. In some embodiments, each component is “pharmaceutically acceptable” in the sense of being compatible with the other ingredients of a pharmaceutical formulation, and suitable for use in contact with the organ or tissue of a subject (e.g., a mammal) without excessive toxicity, allergic response, irritation, immunogenicity, or other complications or problems. See, e.g., Remington: The Science and Practice of Pharmacy, 22nd ed.; Allen Ed.: Philadelphia, P A, 2012; Handbook of Pharmaceutical Excipients, 7th ed.; Rowe et al., Eds.; The Pharmaceutical Press and the American Pharmaceutical Association: 2012; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, FL, 2009.

As used herein, the term related to the percentage of sequence identity such as “at least 70% identical to SEQ ID NO: 31” refers to the extent that sequences are identical on a nucleotide-by-nucleotide basis over a window of comparison. The percentage of sequence identity may be calculated by (1) comparing two optimally aligned sequences over the comparison window; (2) determining the number of positions where the identical nucleic acid base (e.g., U, A, T, C, and G) shows in both sequences to find the number of matched positions; and (3) dividing the number of matched positions by the total number of positions in the comparison window and then multiplying the result thereof by 100 to yield the percentage of sequence identity. In at least one embodiment, included herein are nucleotides having at least about 70%, 75%, 80%, 85%, 90%, 95%, 97%, 98%, 99% or 100% sequence identity to any of the specific sequences (e.g., SEQ ID NO: 31) recited in the Sequence Listing, and these nucleotide variants maintain at least one biological activity or function of the specific sequences.

As used herein, “cell” refers to the smallest structural unit of living matter capable of functioning autonomously, consisting of one or more nuclei, cytoplasm, and various organelles, all surrounded by a semipermeable membrane. Cells include all somatic cells obtained or derived from a living or deceased animal body at any stage of development as well as germ cells, including sperm and eggs (animal reproductive body consisting of an ovum or embryo together with nutritive and protective envelopes). Included are both general categories of cells: prokaryotes and eukaryotes. The cells contemplated for use in this invention include all types of cells from all organisms in all kingdoms: plans, animals, protists, fungi, archaebacteria and eubacteria. Stem cells are cells capable, by successive divisions, of producing specialized cells on many different levels. For example, hematopoietic stem cells produce both red blood cells and white blood cells. From conception until death, humans contain stem cells, but in adults their power to differentiate is reduced.

As used herein, the term “adjuvant” refers to a compound that, with a specific immunogen or antigen, will augment or otherwise alter or modify the resultant immune response. Modification of the immune response includes intensification or broadening the specificity of either or both antibody and cellular immune responses. Modification of the immune response can also mean decreasing or suppressing certain antigen-specific immune responses. In certain embodiments, the adjuvant is a cyclic dinucleotide.

As used herein, the term “amino acid” refers to naturally occurring and synthetic amino acids, as well as amino acid analogs and amino acid mimetics that function in a manner similar to the naturally occurring amino acids.

As used herein, the terms “CpG oligodeoxynucleotide” and “CpG motif” refer to a short single-stranded DNA molecule which includes a 5′ C nucleotide connected to a 3′ G nucleotide through a phosphodiester internucleotide linkage or a phosphodiester derivative internucleotide linkage. In some embodiments, a CpG motif includes a phosphodiester internucleotide linkage. In some embodiments, a CpG motif includes a phosphodiester derivative internucleotide linkage.

As used herein, the terms “coronavirus spike protein” and “coronavirus spike peptide” refer to a full-length or fragment of a large, type 1 transmembrane protein, sometimes referred to as an “S protein,” which includes an S1 and S2 domain. Coronavirus spike proteins are highly glycosylated and assemble in trimers on the virion surface, such as the surface of the SAR-CoV-2 virion.

As used herein, “immune response” refers to a response made by the immune system of an organism to a substance, which includes but is not limited to foreign proteins or self-proteins. Three general types of “immune response” include mucosal, humoral, and cellular immune responses. An immune response may include at least one of the following: antibody production, inflammation, developing immunity, developing hypersensitivity to an antigen, the response of antigen-specific lymphocytes to antigen, and transplant or graft rejection.

As used herein, the term “interleukin (IL)” is a type of cytokine first thought to be expressed by leukocytes alone but have later been found to be produced by many other body cells. As used herein, the term “interferon (IFN)” is a group of signaling proteins made and released by host cells in response to the presence of several viruses. As used herein, the term “tumor necrosis factor (TNF)” is a multifunctional cytokine that plays important roles in diverse cellular events such as cell survival, proliferation, differentiation, and death.

### Example

Exemplary embodiments of the present disclosure are further described in the following examples, which should not be construed to limit the scope of the present disclosure.

Materials and Methods

Mice

Mice were purchased from BioLASCO Taiwan (Taipei, Taiwan) or the National Laboratory Animal Center (Taipei, Taiwan) and were housed at the Laboratory Animal Center of the National Health Research Institutes (NHRI). Mice used in this study were 6-8 weeks old. These mice were maintained and handled following stated guidelines. All procedures were approved by the Institutional Animal Care and Use Committee of the NHRI.

Antigen and Adjuvants

Recombinant SARS-CoV-2 RBD protein (amino acid residues Arg319—Phe541) was purchased from Elabscience (Catalog No. PKSR030521). STING ligand 2′2′-cGAMP (Catalog No. 22419) was from Cayman, and 2′3′-cGAMP (Catalog No. vac-nacga23), 2′3′-cGAM(PS)2 (Catalog No. tlrl-nacga2srs), and 2′3′-c-di-AM(PS)2 (Catalog No. vac-nacda2r) were from InvivoGen. The TLR9 agonist CpG-2722 was synthesized by Integrated DNA Technologies. The adjuvant doses used were 5 μg and 10 μg per mouse for STING ligands and CpG-2722, respectively. Mouse immunization Female BALB/c mice were used for immunogenicity studies. The vaccine was administered in three doses at 10-day intervals. The mice were intramuscularly or intranasally immunized on days 0, 11, and 21 with the formulated RBD/adjuvant vaccines. Control mice received PBS. Intramuscular immunization was done through injections in the right hind leg quadriceps muscle with 50 μl of the antigen/adjuvant mix. For intranasal inoculation, 30 μl of the vaccine was dropped into the nostrils (15 μL for each nostril). Serum samples were obtained 10 days after each immunization for humoral immune response analysis. Spleens, draining lymph nodes, bronchoalveolar lavage fluid (BALF), nasal lavage fluid (NLF), and nasal tissues were collected 10 days after the final vaccination.

Antigen-Dependent Immune Responses and Cytokine Assays

Immunized mice were euthanized 10 days after the final immunization and spleens were isolated for splenocyte preparations. The cells were plated with 6×105 cells/well in 96-well plates (Corning) and stimulated with 10 μg/ml spike RBD protein (Elabscience). After 96 h, supernatants were harvested to analyze cytokine levels. Mouse IL-4, IL-5, IL-13, IL-17, and IFN-γ were measured with commercially available ELISA kits (Invitrogen) according to the manufacturer's protocol.

ELISA for Antibody Responses

Anti-S protein-specific antibody titers in serum, BALF, and NLF samples collected from immunized animals were detected with ELISA. 96-well plates were coated with 4 μg/ml recombinant S protein in PBS at 4° C. overnight. Plates were washed with PBS containing 0.05% Tween-20 and blocked with 1% BSA in PBS for 1 h at room temperature. Serially diluted samples were added and incubated for 2 h at room temperature. Plates were washed and incubated for 1 h at room temperature with HRP-conjugated goat anti-mouse IgG or IgA (Invitrogen). Following washes, signals were developed with TMB substrate (Thermo Scientific) for 15 min, then the colorimetric reaction was stopped with 2 N H2504. The optical density was measured using a microplate reader at a 450 nm wavelength.

hACE2-RBD Competition Assay

96-well plates were coated with ACE2-Fc (2.5 μg/ml, GenScript) overnight at 4° C. The plates were washed and blocked with 1% BSA at 37° C. for 1 h. After washing three times, the plates were incubated with RBD-HRP (1:1000, GenScript) mixed with serially diluted serum samples at 37° C. for 1 h. After washing three times, the signals were developed by incubation with TMB substrate (Thermo Scientific) at 37° C. for 20 min. The reaction was stopped with 2 N H2SO4. Then, the absorbance was measured using a microplate reader at a 450 nm wavelength. Pseudovirus neutralization assay A VSV-Luc-based neutralization assay was performed as previously described. Briefly, 1×104 BHK21-hACE2 cells were seeded in 96-well plates. Heat-inactivated mouse serum samples (56° C. for 30 min) were diluted in PRMI medium. The diluted serum samples were mixed with ˜2×103 pfu VSVΔG-FLuc/SΔ19 pseudovirus at 37° C. for 1 h. Then, the cells were incubated with the serum-virus mixture at 37° C. for 60 min. After 24 h, the luciferase activity in cells was measured with the ONE-Glo™ luciferase assay system (Promega) for infection with pseudovirus.

SARS-CoV-2 Neutralization Assay

A wild-type SARS-CoV-2 neutralization assay was performed in the BSL-3 lab at NHRI. Vero cells were seeded (2.4×104 cells/well) in 96-well plates for 24 h. Heat-inactivated serum (56° C. for 30 min) from mice was serially diluted in M199 medium in 2-fold dilutions from 1:20. The diluted serum was mixed with SARS-CoV-2 virus with 200 TCID50 for 2 h at 37° C. The mixture was added in quadruplicate to the cells, and the cytopathic effect of each well was recorded after 4-5 days of incubation. The neutralization titer of the serum sample was calculated as the reciprocal of the highest serum dilution that prevented viral infection by 50%.

Cell Preparation and Culture

Mouse splenocytes and peripheral blood mononuclear cells (PBMCs) were isolated from C57BL/6J mice and cultured in RPMI 1640 medium supplemented with 10% FBS, 2 mM L-glutamine, 10 mM HEPES, and 1% antibiotic-antimycotic at 37° C. in 5% CO2. To prepare the splenocytes, spleens were collected and mashed through a 70-μm nylon cell strainer (Biologix) and centrifuged at 1200 rpm for 10 min. The cells were incubated with ACK lysis buffer for 5 min at room temperature to lyse the erythrocytes, and this reaction was terminated by adding 5 ml PBS. The splenocytes were washed with PBS and plated at 2.5×106 cells/well in a 12-well plate. Mouse PBMCs were isolated by Ficoll gradient centrifugation following the standard procedure (Ficoll-Paque Premium 1.084, GE Healthcare). Briefly, mouse whole blood was collected in heparinized tubes (BD Biosciences) and diluted with up to 4 ml PBS. The diluted blood was carefully layered on Ficoll-Paque Premium (3 ml). The samples were centrifuged at 400×g at 20° C. for 40 min. The PBMC-containing interphase layer was removed, washed twice with PBS, and subsequently plated at 1×106 cells/well in a 12-well plate. Mouse embryonic fibroblasts (MEFs) were prepared from C57BL/6J mice. The cells were seeded at 4×105 cells/well in a 12-well plate in DMEM medium supplemented with 10% FBS, 2 mM L-glutamine, 1 mM sodium pyruvate, 10 mM HEPES, and 1% antibiotic-antimycotic at 37° C. in 5% CO2. Bone marrow derived macrophages (BMDCs) were prepared from C57BL/6J mice. Bone marrow cells were flushed from the mouse femur and tibia with PBS followed by red blood cell lysis and PBS wash, and then grown in culture medium (RPMI 1640 supplemented 10% FBS, 2 mM L-glutamine, 1% antibiotic-antimycotic, 1% MEM nonessential amino acid solution, 1 mM sodium pyruvate, 10 mM HEPES) containing 100 ng/mL FMS-like tyrosine kinase 3 ligand (Flt3-L) (PeproTech) and 5 ng/mL granulocyte-macrophage colony-stimulating factor (GM-CSF) (PeproTech). At days 3 and 6, additional culture medium containing 100 ng/mL Flt3-L and 5 ng/mL GM-CSF was added to the plate. At day 9, the non-adherent cells were collected and seeded at 2×106 cells/well of a noncoated six-well plate.

Flow Cytometry

Spleens and draining lymph nodes were collected from the vaccinated mice, and single-cell suspensions were prepared, washed with FACS buffer (PBS containing 2% FBS), and maintained in the dark at 4° C. The viable cells were stained with Fixable Viability Stain 620 (BD Biosciences) and were then incubated with anti-CD16/32 (S17011E, BioLegend) for 10 min to reduce nonspecific antibody binding. After Fc blocking, the cell suspensions were stained for 30 min with antibodies to B and T cell surface markers: FITC-GL7 (GL7, BioLegend), PE-CD38 (90, eBioscience), APC-Cy7-B220 (RA3-6B2, BioLegend), BV421-CD95 (Jo2, BD Biosciences), BV510-CD19 (1D3, BD Biosciences), BB515-ICOS (7E.17G9, BD Biosciences), PE-CXCR5 (SPRCLS, eBioscience), BV421-CD4 (GK1.5, BioLegend), and BV510-PD-1 (J43, BD Biosciences). For intracellular staining, T cells were fixed with the Transcription Factor Buffer Set according to the manufacturer's instructions (BD Biosciences). The cells were then intracellularly stained with Alexa Fluor 647-Bcl-6 (IG191E/A8, BioLegend) for 50 min. For BMDCs, cells were stained with PE-CD11c (N418, Invitrogen), Pacific Blue-CD40 (1C10, Invitrogen), APC-CD80 (16-10A1, Invitrogen),

PE-Cy7-CD86 (GL1, Invitrogen), and APC-Cy7-CCR7 (4B12, Invitrogen) for 30 min. Fluorescence was analyzed using a FACSCanto II Flow Cytometer (BD Biosciences). Data were analyzed using FlowJo software.

TABLE 1
List of qPCR primer sequences used in this study (SEQ ID NO: 1-30)

| Gene | Forward | Reverse | SEQ ID NO |
|---|---|---|---|
| IL-1b | AGAGCTTCAGGCAGGCAGTA | AGGTGCTCATGTCCTCATCC | 1, 2 |
| IL-6 | CCGGAGAGGAGACTTCACAG | TTTCCACGATTTCCCAGAGA | 3, 4 |
| IL-8 | CATTTGGGAGACCTGAGAACA | TGGAGTCCCGTAGAAAATTCC | 5, 6 |
| IL-12A | ACGGCCAGAGAAAAACTGAA | CTACCAAGGCACAGGGTCAT | 7, 8 |
| IL-12B | CACGCCTGAAGAAGATGACA | AGTCCCTTTGGTCCAGTGTG | 9, 10 |
| IL-23A | CATGCTAGCCTGGAACGCACAT | ACTGGCTGTTGTCCTTGAGTCC | 11, 12 |
| TNF-a | ACGGCATGGATCTCAAAGAC | GTGGGTGAGGAGCACGTAG | 13, 14 |
| IFN-a | ATCCAGAAGGCTCAAGCCATCC | GGAGGGTTGTATTCCAAGCAGC | 15, 16 |
| IFN-b | GCCTTTGCCATCCAAGAGATGC | ACACTGTCTGCTGGTGGAGTTC | 17, 18 |
| IFN-g | CAGCAACAGCAAGGCGAAAAAGG | TTTCCGCTTCCTGAGGCTGGAT | 19, 20 |
| IL-2 | GCGGCATGTTCTGGATTTGACTC | CCACCACAGTTGCTGACTCATC | 21, 22 |
| IL-4 | ATCATCGGCATTTTGAACGAGGTC | ACCTTGGAAGCCCTACAGACGA | 23, 24 |
| IL-5 | GATGAGGCTTCCTGTCCCTACT | TGACAGGTTTTGGAATAGCATTTCC | 25, 26 |
| IL-13 | AACGGCAGCATGGTATGGAGTG | TGGGTCCTGTAGATGGCATTGC | 27, 28 |
| beta-Actin | CATTGCTGACAGGATGCAGAAGG | TGCTGGAAGGTGGACAGTGAGG | 29, 30 |

RNA Isolation and Reverse Transcription-Quantitative PCR

Total RNA was extracted from mouse BMDCs, splenocytes, PBMCs, and MEFs using the illustra RNAspin Mini Kit (GE Healthcare) according to the manufacturer's protocol. First-strand cDNA was synthesized from total RNA in the presence of random hexamers using the SuperScript IV First-Strand Synthesis System (Invitrogen). Quantitative PCR was performed with the QuantiNova SYBR Green PCR Kit (Qiagen) using Applied Biosystems ViiA 7 Real-Time PCR System with gene-specific primers (Table 1) for gene expression analysis. All primers used were synthesized by Protech Technology (Taipei, Taiwan). Target gene expression was calculated by the comparative ΔΔcycle threshold (Ct) method for relative quantification after normalization to ACTB expression.

Hematoxylin and Eosin (H&E) Staining

Mice were sacrificed 10 days after the final vaccination. The nasal tissues were harvested and fixed with 10% buffered formalin. Paraffin-embedded tissues were sectioned into 5-μm tissue slides. These tissue slides were stained with H&E by the Pathology Core Laboratory of NHRI for histological examination.

Statistical Analysis

Data are presented as the mean±SEM from at least three independent experiments. Statistical significance was determined by the unpaired Student's t-test. P<0.05 was considered statistically significant.

Results

Adjuvant Activities of CpG-2722 and STING Agonist Combinations for Intramuscularly and Intranasally Administrated SARS-CoV-2 RBD Vaccine

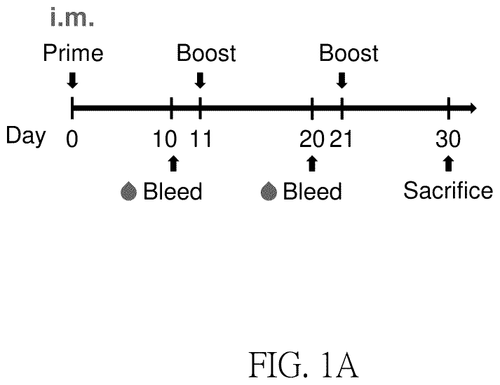

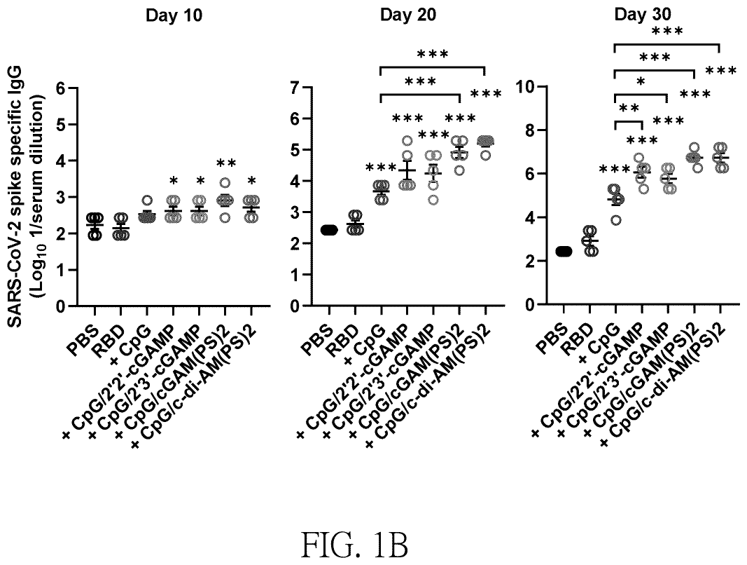

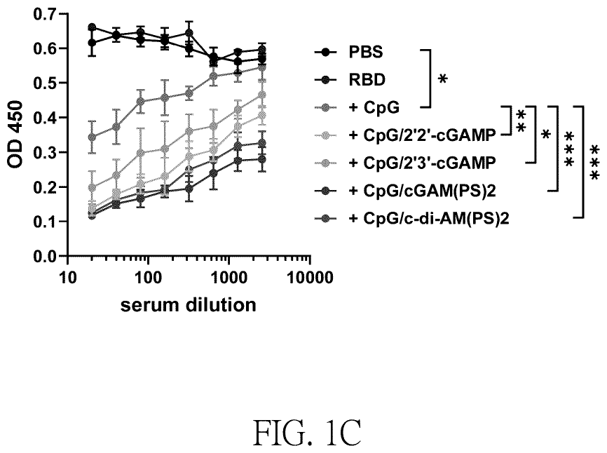

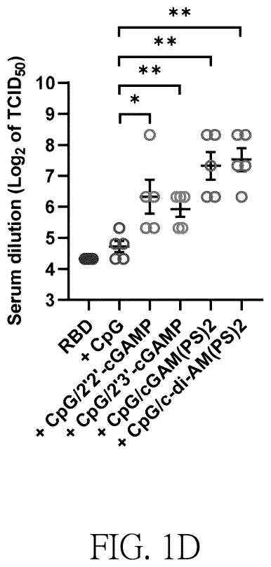

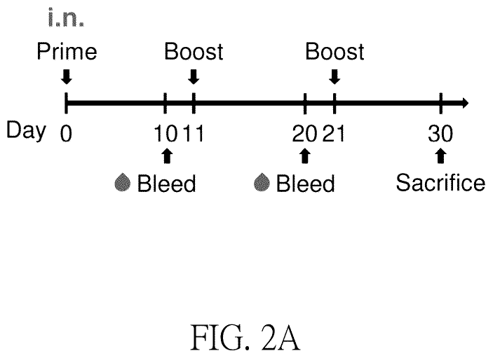

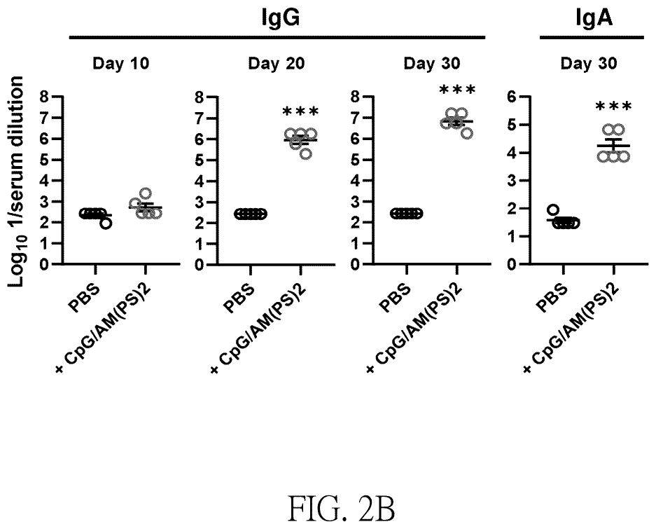

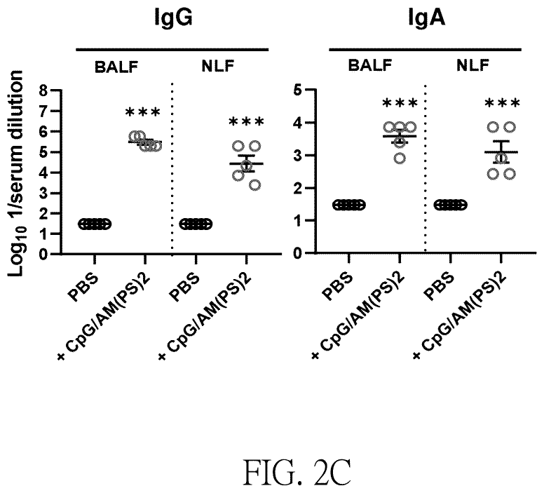

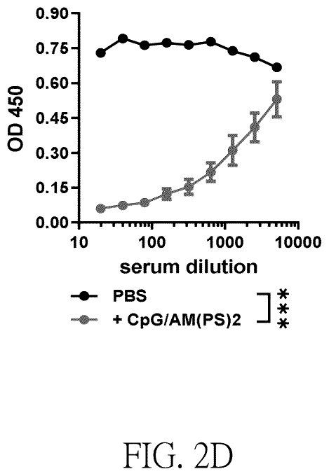

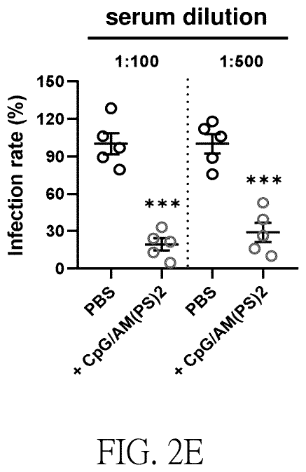

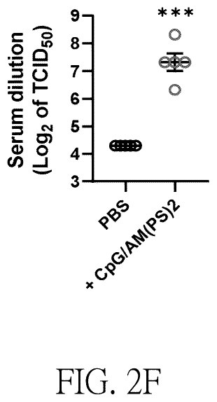

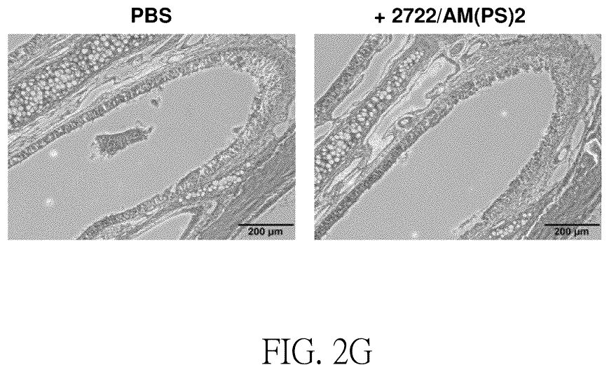

CpG-2722 is a CpG-ODN with 19 nucleotides containing two GTCGTT hexamer CpG-motifs and four thymidine bases between these two motifs. The STING agonist 2′2′-cGAMP is a synthetic CDN containing two 2′-5′-phosphodiester bonds between the guanosine and adenosine, and 2′3′-cGAMP is a mammalian CDN containing 2′-5′ and 3′-5′ phosphodiester linkages between the guanosine and adenosine. 2′3′-cGAM(PS)2 is a bisphosphorothioated analog of 2′3′-cGAMP, and 2′3′-c-di-AM(PS)2 is a bisphosphorothioated analog of 2′3′-c-di-AMP. To evaluate the adjuvant activities of combined CpG-2722 and CDNs, recombinant SARS-CoV-2 RBD protein was formulated as an antigen with/without CpG-2722 alone or in combination with different CDNs. BALB/c mice were intramuscularly immunized with these vaccines, and blood samples were collected in 10 days intervals to analyze antibody titers and anti-viral responses following the schedule in FIG. 1A. As shown in FIG. 1B, CpG-2722/CDN adjuvanted vaccines increased anti-S protein (hereafter anti-S) IgG titer on day 10 after the first inoculation. On day 20, after the 2nd injection, CpG-2722 and CpG-2722/CDN combinations induced a significantly higher anti-S protein IgG titer than that induced by the vehicle control. Additionally, the CpG-2722/2′3′-cGAM(PS)2 and CpG-2722/2′3′-c-di-AM(PS)2 formulations enhanced anti-S IgG titer compared with CpG-2722 alone. These enhanced effects were also apparent for the CpG-2722/CDN formulations after the 3rd vaccination (FIG. 1B). Next, we analyzed the abilities of the serum samples from the immunized mice to block the interaction between RBD and ACE2 and neutralize the SARS-CoV-2 virus (hCoV-19/Taiwan/4/2020) infection in Vero E6 cells. Consistently, CpG-2722/CDN adjuvanted vaccines generated higher antibody titers for interfering with the RBD protein/ACE2 interaction and inhibition of viral infection than that generated by the vaccine adjuvanted with CpG-2722 alone (FIG. 1C and FIG. 1D). The inhibition dilution of TCID50 (Median Tissue Culture Infectious Dose) for serum from the CpG-2722/2′2′-cGAMP, CpG-2722/2′3′-cGAMP, CpG-2722/2′3′-cGAM(PS)2, CpG-2722/2′3′-c-di-AM(PS)2 groups were 112, 64, 192, and 224, respectively (FIG. 1D). The adjuvant activity of the CpG-2722/2′3′-c-di-AM(PS)2 combination for intranasal immunization was investigated with a 10-days interval of nasal administration and blood sample collection schedule (FIG. 2A). The CpG-2722 and 2′3′-c-di-AM(PS)2 combination robustly induced an antibody response to the SARS-CoV-2 S protein. Anti-S IgG was detected in the 106-fold diluted serum collected after the second immunization. Furthermore, anti-S IgG and IgA were detected in the 107 and 104-fold diluted serum on day 30, respectively (FIG. 2B). The levels of anti-S IgG and IgA in BALF and NLF after the third immunization were also investigated. Consistently, the CpG-2722/2′3′-c-di-AM(PS)2 adjuvanted vaccine generated considerable amounts of S protein-specific IgG and IgA in the bronchoalveolar and nasal cavities (FIG. 2C). The collected sera from the immunized mice contained RBD neutralization antibodies to block the interaction between RBD and ACE2 (FIG. 2D). The capability of these sera to interfere with the entrance of the S protein carrying vesicular stomatitis virus (S-VSV) pseudoviruses into hACE2-expressing BHK21 cells was investigated. The results revealed that 80.9% and 69.2% of the pseudovirus were inhibited by sera dilutions of 100 and 500, respectively (FIG. 2E). Furthermore, in the SARS-CoV-2 neutralization assay, the sera showed an inhibition dilution of 176 for TCID50 (FIG. 2F). Following immunizations, the mouse nasal cavities were histologically examined. No indication of pathological tissue damage or inflammation was found (FIG. 2G). Taken together, these results demonstrate the potential of the CpG-2722/2′3′-c-di-AM(PS)2 combination as an adjuvant for muscle and nasal-delivered vaccines.

Induction of Germinal Center B Cell Response by Combinations of CpG-2722 and Different STING Agonists

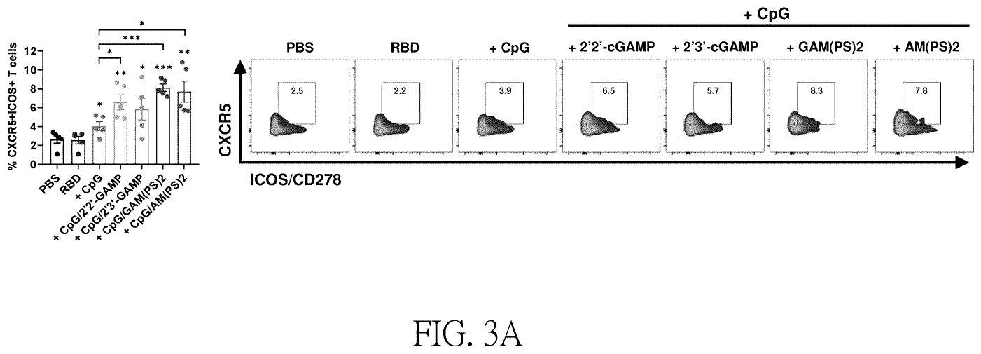

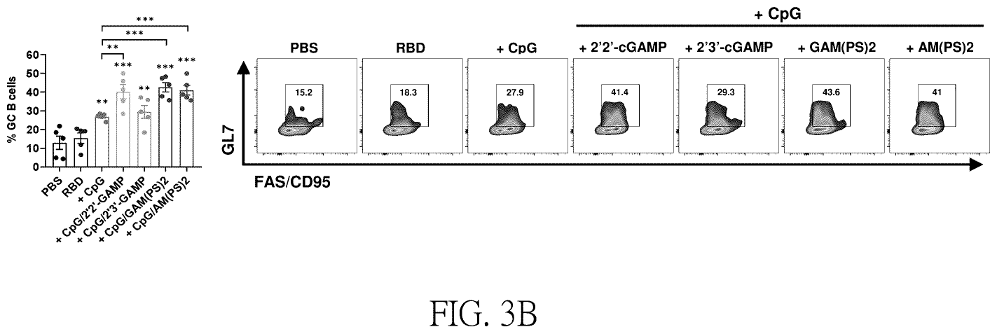

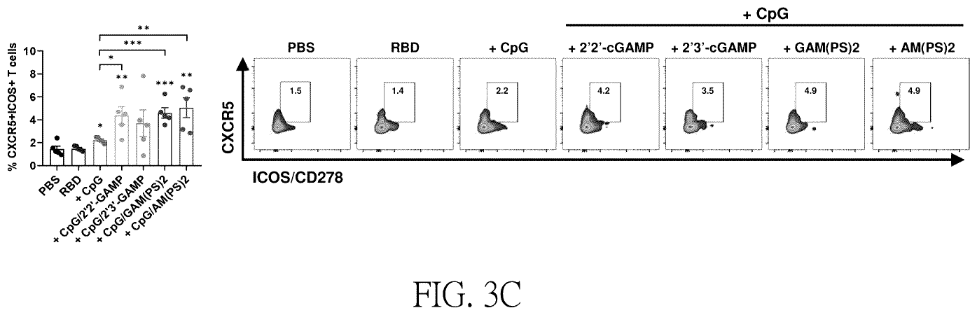

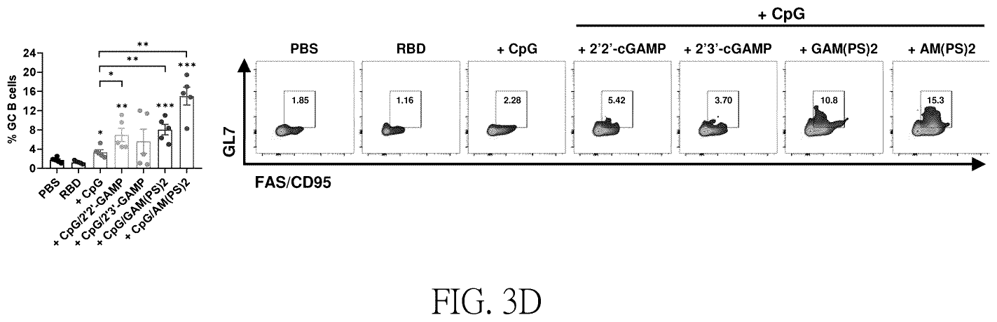

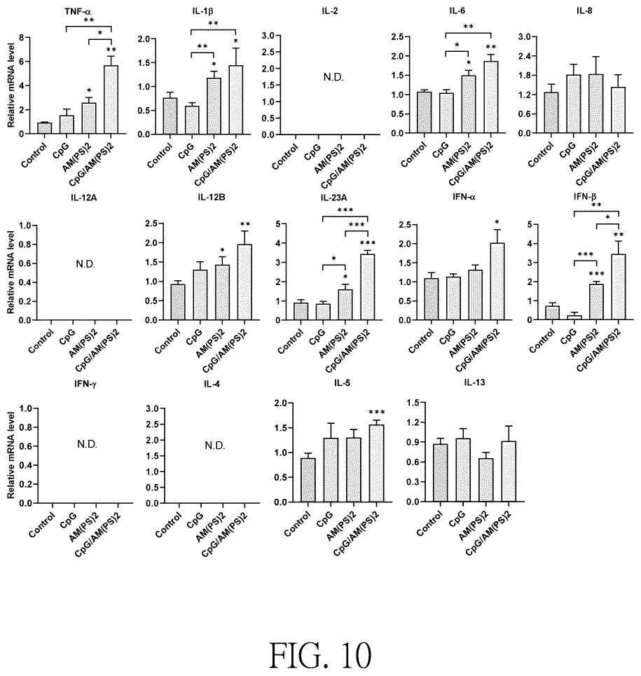

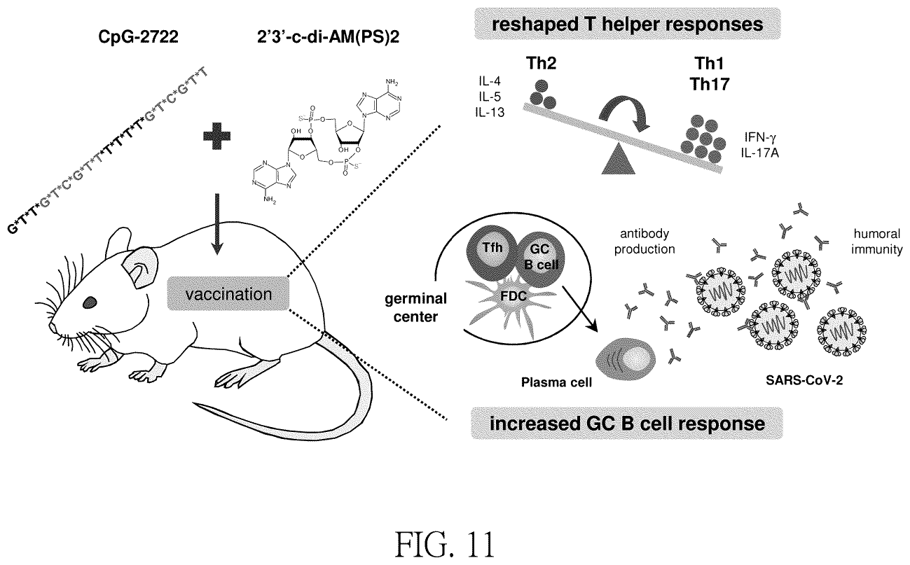

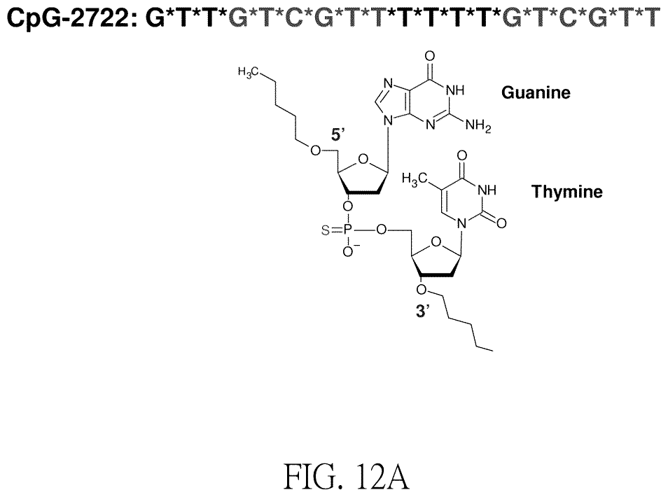

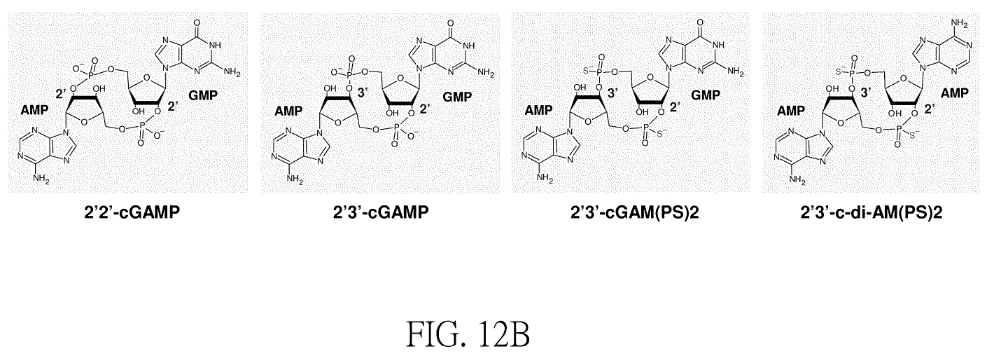

Germinal center (GC) response is critical for the generation of mature plasma cells and memory B cells for long-lasting protective immunity. T follicular helper (Tfh) cells play a key role in regulating the GC response. To assess the effects of CpG-2722/CDN combined adjuvants on the induction of a GC response, the vaccinated mice in the experiment for FIG. 1 were euthanized after the final serum collection. Draining lymph node (dLN) cells and splenocytes were isolated for flow cytometry analysis of the CXCR5+/ICOS+ and CXCR5+/PD-1+Tfh cells and the GL7+/CD95+GC B cells. The results showed that the CpG-2722/2′2′-cGAMP, CpG-2722/2′3′-cGAM(PS)2, and CpG-2722/2′3′-c-di-AM(PS)2 adjuvanted vaccines increased the Tfh and GC B cell counts in dLNs and spleens compared to the CpG-2722-adjuvanted vaccine (FIG. 3A, FIG. 3B, FIG. 3C; FIG. 3D; and FIG. 12A, and FIG. 12B). Additionally, except for the CpG-2722/2′3′-cGAMP combination, the activities of these CpG-2722/CDN combinations on the induction of a GC B cell response were consistent with the humoral response shown in FIG. 1.

CpG-2722 and c-Di-AM(PS)2 Cooperatively Boost Immune Responses to the SARS-CoV-2 RBD Vaccine

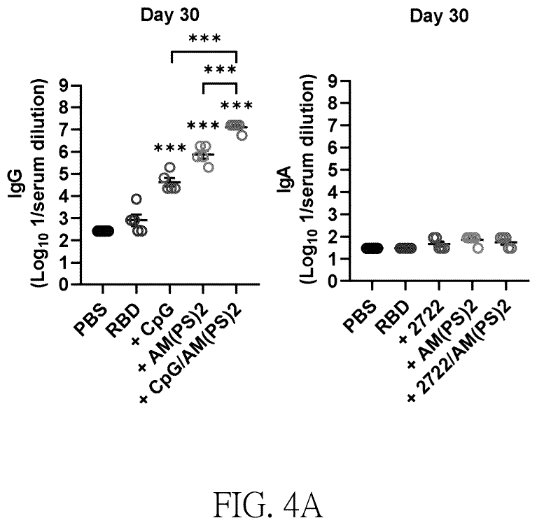

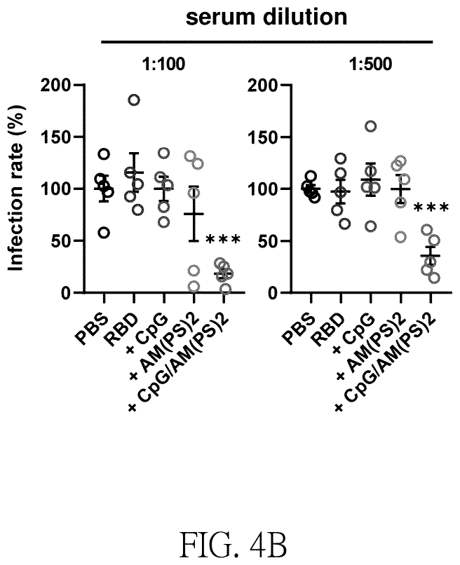

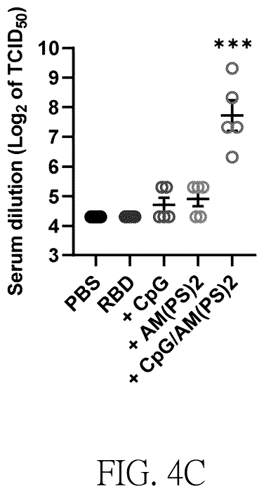

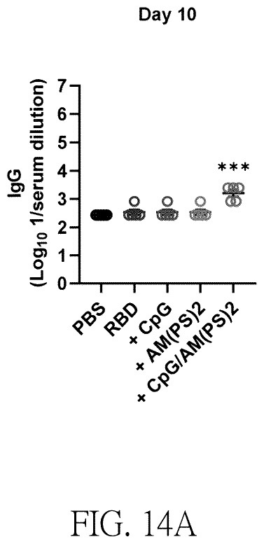

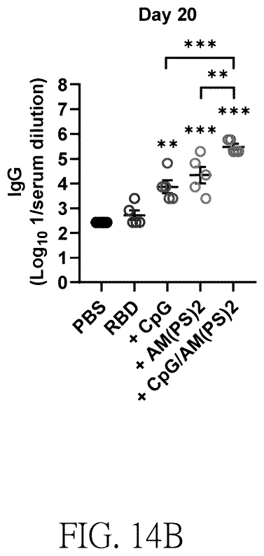

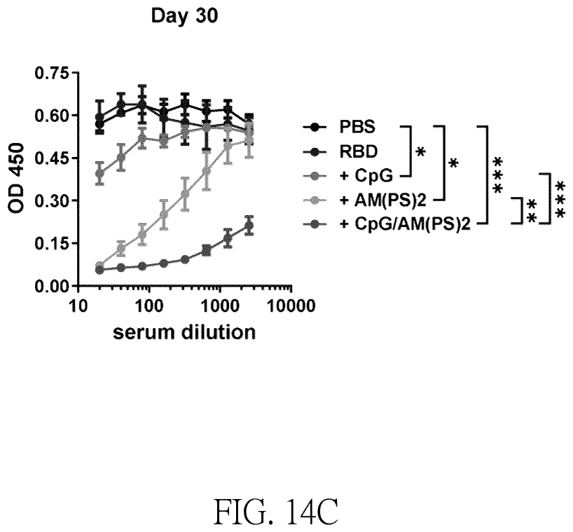

2′3′-c-di-AM(PS)2 (hereafter c-di-AM(PS)2) was used to study the molecular basis for the adjuvant activity of the CpG-2722 and STING agonist combination. Whether the increased adjuvant effect of CpG-2722/c-di-AM(PS)2 came from a cooperative effect of these two agonists was first investigated. SARS-CoV-2 RBD protein was formulated with or without CpG-2722 and c-di-AM(PS)2 alone or in combination. Mice were vaccinated with a 10-day interval intramuscular immunization and blood collection schedule, as shown in FIG. 1A. On day 10 after the first inoculation, anti-S IgG was detected in the group that received the CpG-2722/c-di-AM(PS)2 adjuvanted vaccine, but not in the groups that received the CpG-2722 or c-di-AM(PS)2 alone adjuvanted vaccine (FIG. 14A). On day 20, after the 2nd injection, the vaccine adjuvant activities of CpG-2722 and c-di-AM(PS)2 became apparent, and the CpG-2722/c-di-AM(PS)2 combination induced a significantly higher anti-S protein IgG titer than that induced by the CpG-2722 or c-di-AM(PS)2 alone (FIG. 14B). On day 30, after completing the entire immunization process, the immune sera from different groups were examined for the levels of anti-S IgG and anti-S IgA. The vaccine adjuvanted with the CpG-2722/c-di-AM(PS)2 combination continued to induce a higher anti-S IgG titer than that induced by the CpG-2722 or the c-di-AM(PS)2 adjuvanted vaccines. In contrast, the vaccines adjuvanted with CpG-2722 and c-di-AM(PS)2 alone or in combination did not elicit the production of serum anti-S IgA (FIG. 4A). This was different from that seen in FIG. 2B for the induction of anti-S IgA by the intranasally administrated vaccine. These sera from different adjuvanted vaccine groups contained neutralization antibodies that blocked the binding of RBD protein to hACE2. Moreover, sera from the CpG-2722/c-di-AM(PS)2 adjuvanted group had a stronger blocking activity than that from the CpG-2722 or the c-di-AM(PS)2 alone adjuvanted groups (FIG. 14C). The capability of these sera to interfere with the entrance of S-VSV pseudoviruses into hACE2-expressing BHK21 cells and to neutralize infection with SARS-CoV-2 viruses into Vero cells were investigated. The results revealed 81.9% and 71.8% blocking of the pseudovirus infection by the sera from the CpG-2722/c-di-AM(PS)2 adjuvanted vaccine group at 100 and 500-fold dilution, respectively (FIG. 4B). Additionally, in the SARS-CoV-2 neutralization assay, the sera showed an inhibition dilution of 208 for TCID50 (FIG. 4C). Furthermore, in both studies, sera from the CpG-2722/c-di-AM(PS)2 adjuvanted vaccine group showed stronger neutralization activity compared to that of the CpG-2722 or the c-di-AM(PS)2 adjuvanted vaccine groups (FIG. 4B and FIG. 4C). These results suggest a cooperative adjuvant effect between the TLR9 and the STING agonists on boosting the humoral immune response to the vaccine.

Antigen-Dependent T Helper Responses Induced by Vaccines Adjuvanted with CpG-2722 and c-Di-AM(PS)2 Alone and in Combination

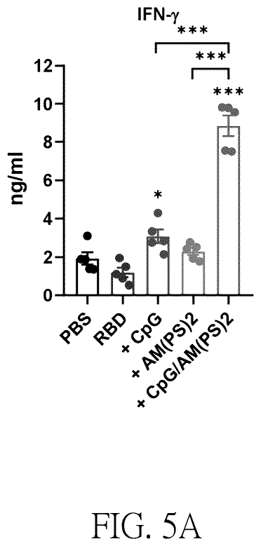

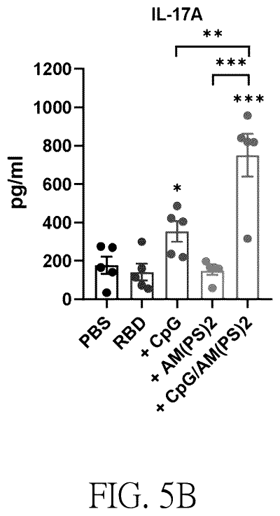

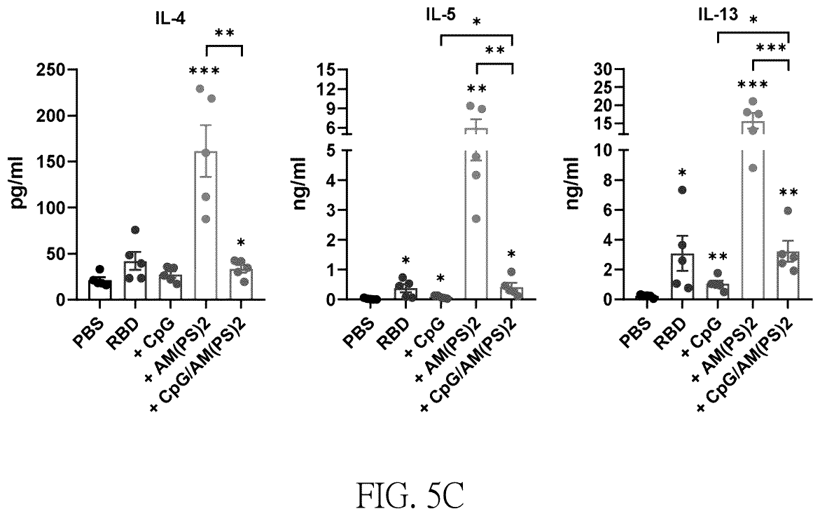

At the endpoint of the experiment in FIG. 4, splenocytes from a different group of mice were collected to investigate antigen-dependent Th responses. These cells were stimulated with the RBD protein antigen and the production of signature cytokines for different Th responses were measured with ELISA. These analyzed cytokines were: IFN-γ for Th1, IL-17A for Th17, and IL-4, IL-5, and IL-13 for Th2 response. The results showed that CpG-2722 adjuvanted vaccine preferentially induced antigen-dependent Th1 and Th17 responses in the immunized mice, whereas the c-di-AM(PS)2 adjuvanted vaccine induced an antigen-dependent Th2 response. Furthermore, compared to that induced by the CpG-2722 and c-di-AM(PS)2 adjuvanted vaccines, combining CpG-2722 and c-di-AM(PS)2 in a vaccine reshaped the Th response profile in which the Th1 and Th17 responses were synergistically enhanced and the Th2 response was suppressed but compared to the vehicle control, remained significantly activated (FIG. 5A, FIG. 5B, and FIG. 5C).

Activation of Dendritic Cells by CpG-2722, c-Di-AM(PS)2 Alone and their Combination

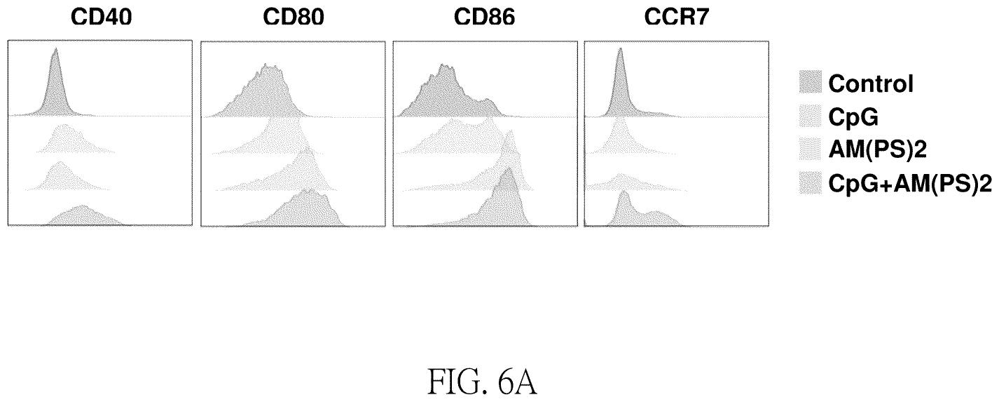

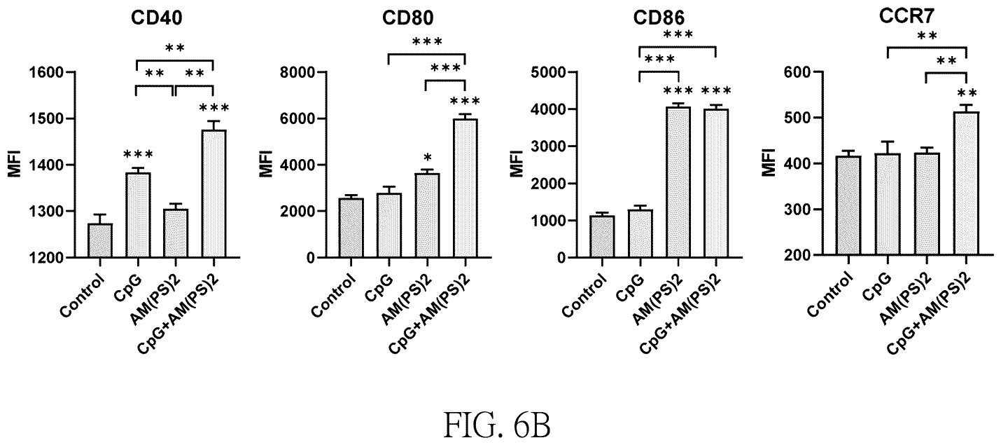

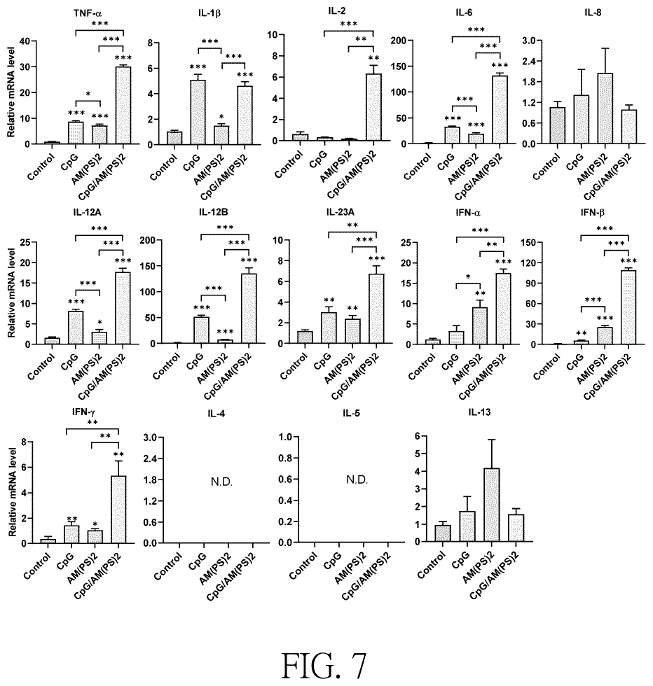

Dendritic cells (DCs) are professional antigen presenting cells and their activation for antigen presentation and cytokine production is critical for effective vaccination and adjuvant-activated antigen dependent T cell responses. Therefore, we investigated the capability of CpG-2722, c-di-AM(PS)2, and their combination to induce the expression of cell surface molecules and cytokines that serve various functions in the activation and maturation of DCs. The expressions of cell surface molecules on bone marrow-derived dendritic cells (BMDCs) were analyzed by flow cytometry following different treatments. CpG-2722 alone induced expression of CD40, c-di-AM(PS)2 alone induced the expression of CD80 and CD86, while none of these two agonists activated expression of the CCR7. The combination of CpG-2722 and c-di-AM(PS)2 displayed a cooperative effect in increasing the expression of CD40, CD80, and CCR7, but the c-di-AM(PS)2 induced CD86 expression was not further increased when combined with CpG-2722 (FIG. 6A and FIG. 6B). CD40, CD80, and CD86 are markers for DC activation and maturation. They are co-stimulatory molecules that promote T cell activation. The CCR7 is a chemokine receptor that plays a critical role in DC migration to lymphoid organs to activate T cells. The activities of CpG-2722, c-di-AM(PS)2, and their combination in inducing cytokine expression in BMDCs were investigated by RT-qPCR. Both CpG-2722 and c-di-AM(PS)2 alone induced the expressions of TNF-α, IL-1β, IL-6, IL-12A, IL-12B, IL-23A, IFN-β, and IFN-γ genes. c-di-AM(PS)2 alone induced the expression of IFN-α. Except for IL-1β, which was strongly induced by the CpG-2722, the combination of CpG-2722 and c-di-AM(PS)2 showed a cooperative effect on inducing the expression of all other cytokines. Notably, the CpG-2722/c-di-AM(PS)2 combination induced the expression of IL-2, which was not induced by either of these two agonists alone (FIG. 7). IL-2 production in DCs has been shown to play an important role in DCs-derived T cell activation.

Antigen-Independent Cytokine-Inducing Profiles of CpG-2722 and c-Di-AM(PS)2 Alone and in Combination in Different Cell Types

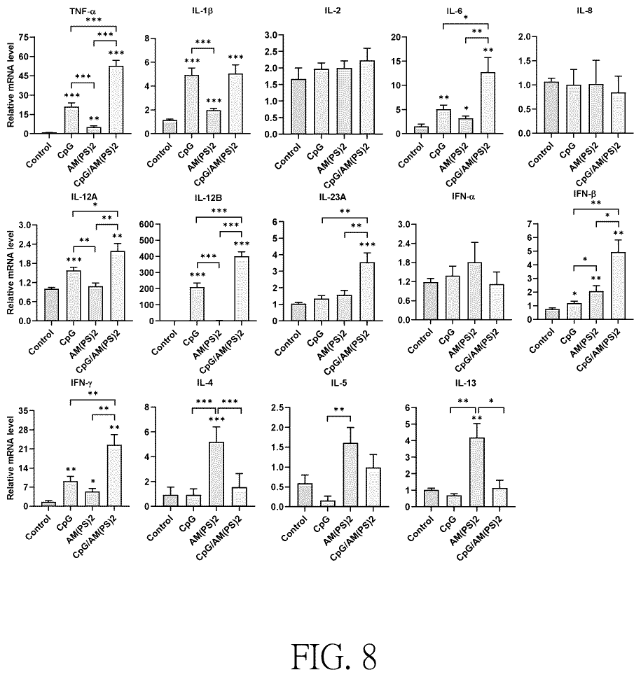

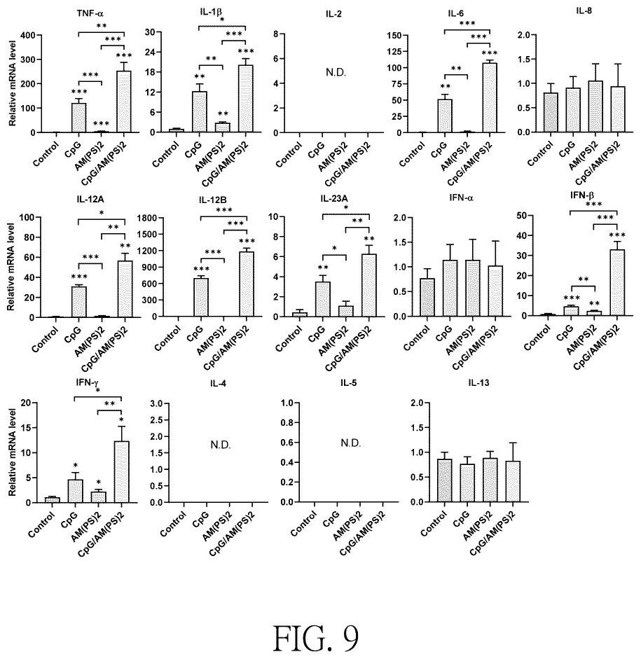

To further gain insight into the mechanism by which antigen-dependent Th responses are shaped by different adjuvants in immunized mice, the activities of CpG-2722, c-di-AM(PS)2 alone and in combination to induce the expression of cytokines in different cell populations were investigated. In splenocytes, CpG-2722 was more potent than c-di-AM(PS)2 in inducing the expression of TNF-α, IL-1β, IL-6, IL-12 A, IL-12B, and IFN-γ. Among these cytokines, IL-12A and IL-12B were not induced by c-di-AM(PS)2 alone. In contrast, c-di-AM(PS)2 induced the expression of IFN-β, IL-4, IL-5, and IL-13 more potently. Furthermore, the combination of CpG-2722 and c-di-AM(PS)2 had a cooperative effect on inducing the expression of TNF-α, IL-6, IL-12A, IL-12B, IL-23A, IFN-β, and IFN-γ, but the expression of IL-4, IL-5, and IL-13 were suppressed with this combination (FIG. 8). Like in splenocytes, the mRNA expression of TNF-α, IL-1β, IL-6, IL-12A, IL-12B, IL-23A, IFN-β as well as IFN-γ in PBMCs showed a higher induction by CpG-2722 treatment alone than that induced by the c-di-AM(PS)2 treatment alone. The combination of these agonists had a cooperative effect on inducing the expression of these cytokines. However, in PBMCs, the expression levels of IL-2, IL-4, and IL-5 were too low to be detected, and the expression of IL-13 was not increased by any stimulation (FIG. 9). In MEFs, cytokine expression activation by CpG-2722 was not observed. In contrast, c-di-AM(PS)2 or the CpG-2722/c-di-AM(PS)2 combination induced the gene expression of TNF-α, IL-1β, IL-6, IL-12B, IL-23A, and IFN-β. Among these genes, TNF-α, IL-23A, and IFN-β expression was further enhanced with CpG-2722/c-di-AM(PS)2 combinational stimulation compared with c-di-AM(PS)2 alone (FIG. 10). These results obtained from BMDCs, splenocytes, PBMCs, and MEFs show that CpG-2722 and c-di-AM(PS)2 have different targeting cell populations and thus have different cytokine-inducing profiles in different cell populations. TNF-α and IFN-γ induced by CpG-2722 are required for Th1 cell differentiation and the activation of IL-1, IL-6, and IL-23 are essential for Th17 cell development. In contrast, c-di-AM(PS)2 preferentially induces IL-4, which plays a key role in the activation of Th2 cell differentiation. These antigen-independent responses shaped the antigen-dependent responses induced by the vaccines adjuvanted with CpG-2722 and c-di-AM(PS)2 alone or in combination. Overall, the increased targeting cell populations, enhanced germinal center B cell response, and the reshaped T helper responses are the molecular bases for the cooperative adjuvant effect of the combination of TLR9 and STING agonists (FIG. 11).

Combination of CpG-2722 and STING Ligands Elicited Robust Tfh Cell Responses.

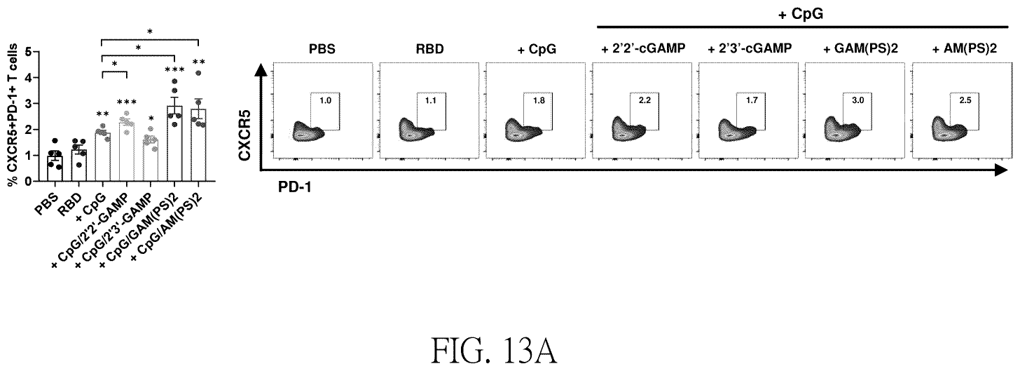

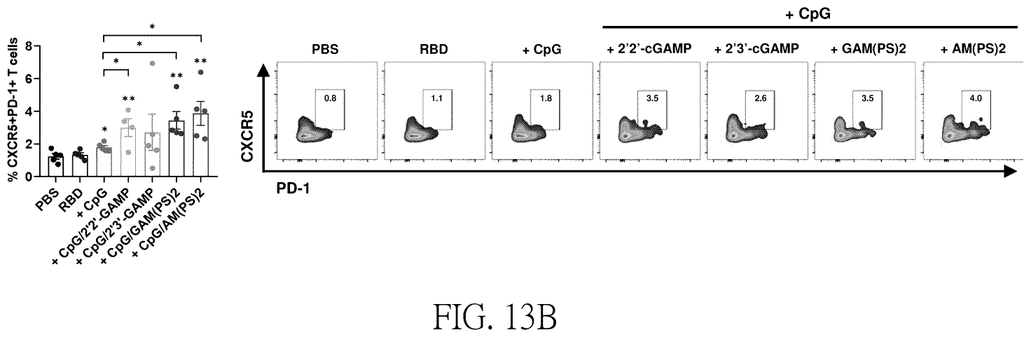

As shown in FIG. 12A, CpG2722 (GTTGTCGTTTTTTGTCGTT, SEQ ID NO: 31) is a TLR9 agonist with a phosphothioated backbone containing 19 nucleotides and two copies of a GTCGTT hexamer motif. Asterisks stand for phosphorothioate bonds. The hexamer motifs are shown in red font. As shown in FIG. 12B, cyclic-dinucleotides (CDNs) for the activation of STING. cGAMP molecules are hybrid CDNs comprising a guanosine monophosphate (GMP) and an adenosine monophosphate (AMP), whereas c-di-AMPs are homodimers of AMP. These CDNs are cyclic by 2′-5′-phosphodiester bonds from the 2′2′ or 2′3′ positions of the two nucleotides as shown. In contrast with the 2′2′-cGAMP and 2′3′-cGAMP, the 2′3′-cGAM(PS)2 and 2′3′-cdi-AM(PS)2 contain two phosphorothioate modifications as shown in red font. In the experiments in FIG. 1, the mice were euthanized 10 days after the final vaccination (day 30) to collect draining lymph nodes and spleens. The numbers of Tfh cells (CD4+Bcl-6+CXCR5+PD-1+) from lymph nodes (shown in FIG. 13A) and spleens (shown in FIG. 13B) were measured by flow cytometry. Data are the mean±SEM (n=5/group).

Cooperative Adjuvant Effect of CpG-2722 and c-Di-AM(PS)2 on Inducing a Humoral Response to the RBD Protein Vaccine.

BALB/c mice were immunized intramuscularly on days 0, 11, and 21 with 10 mg of the RBD protein vaccine adjuvanted with 10 g CpG-2722 and 5 g c-di-AM(PS)2 alone or in combination. Serum samples were collected on days 10, 20, and 30. These samples collected on day 10 (shown in FIG. 14A) and day 20 (shown in FIG. 14B) were analyzed for anti-S protein IgG levels by ELISA. As shown in FIG. 14C, Serum samples collected on day 30 were subjected to the hACE2-RBD competition assay. Data are the mean±SEM (n=5/group).

Adjuvant Activities of CpG-2722 and 2′3′-c-Di-AMP or 2′3′-c-Di-AM(PS)2 Combinations for Intranasally Administrated Influenza Viral Like Particle Vaccines.

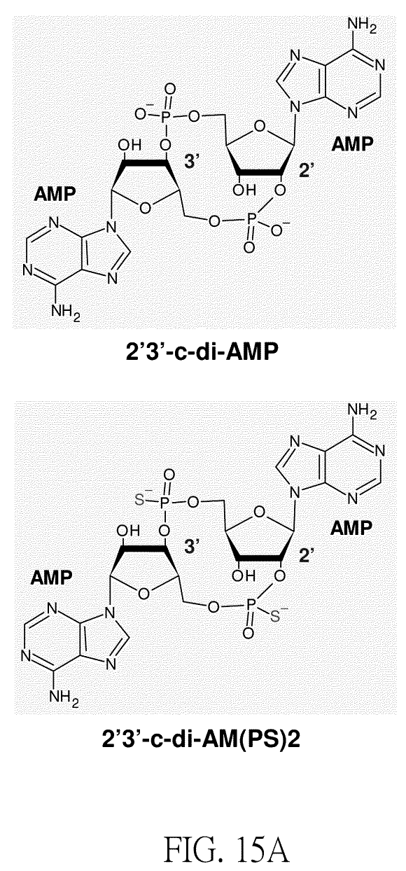

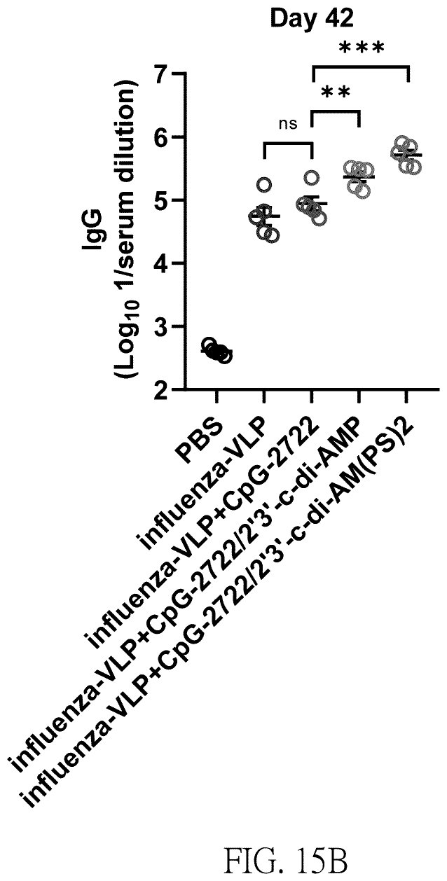

The STING agonist 2′3′-c-di-AMP is a synthetic cyclic di-nucleotides (CDN) that comprises two adenosines linked by 2′-3′-phosphodiester bonds, while 2′3′-c-di-AM(PS)2 is a bisphosphorothioated analog of 2′3′-c-di-AMP (shown in FIG. 15A). We demonstrated the effectiveness of the CpG-2722/2′3′-c-di-AM(PS)2 combination as an adjuvant for SARS-CoV-2 RBD vaccines. To explore the applicability of this combination to different vaccine types and compare the effect of 2′3′-c-di-AMP and 2′3′-c-di-AM(PS)2, we utilized CpG-2722 alone, as well as its combination with these two CDNs respectively, as adjuvants in influenza VLP (viral like particle) vaccines. We conducted intranasal immunizations of BALB/c mice on days 0, 15, and 29 using the formulated VLP/adjuvant vaccines, and blood samples were collected on days 14, 28, and 42 for the analysis of antibody titers. As shown in FIG. 15B, administration of VLP resulted in an increase in anti-VLP IgG titer after the 3rd vaccination (day 42). Because the VLP itself already has good antigenicity, adjuvanted with CpG-2722 alone does not further increase the IgG titer. However, vaccines adjuvanted with CpG-2722/2′3′-c-di-AMP and CpG-2722/2′3′-c-di-AM(PS)2 combinations exhibited enhanced anti-VLP titers compared to the VLP group. The impact of these adjuvants demonstrated a similar effect on IgA antibody titers (shown in FIG. 15C). These results indicate that the combination of CpG-2722 with 2′3′-c-di-AM(PS)2 and the combination of CpG-2722 with 2′3′-c-di-AMP have good adjuvant effects to boost the VLP vaccine responses.

Cooperative Effect of CpG-2722 and 2′3′-c-Di-AMP on the Suppression of Head and Neck Tumor Growth.

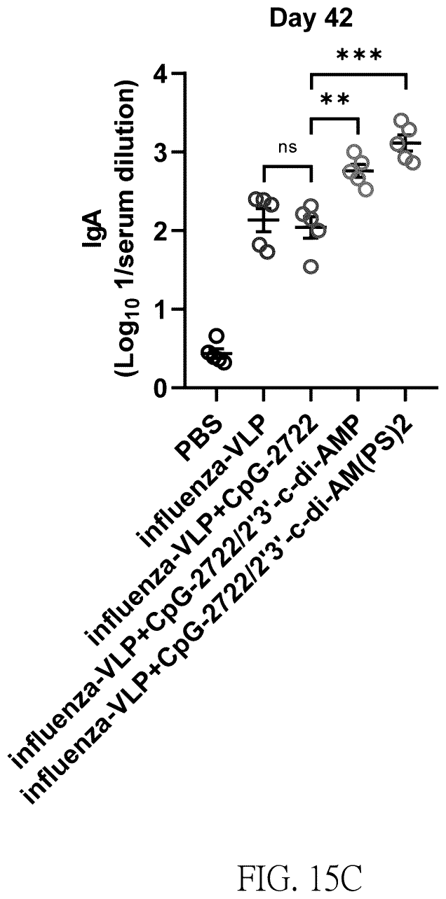

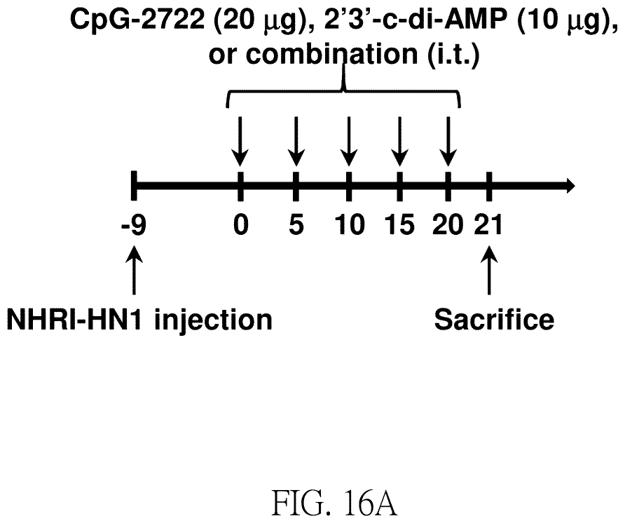

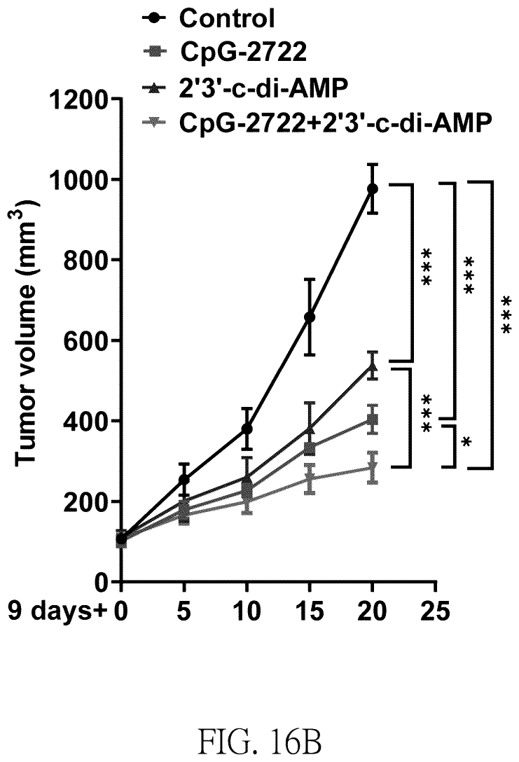

We investigated the combined effect of CpG-2722 and 2′3′-c-di-AMP on suppressing tumor growth using the NHRI-HN1 syngeneic orthotopic cancer animal model. Tumors were allowed to grow for 9 days until they reached approximately 100 mm 3 in size. Subsequently, the mice received intratumoral injections of CpG-2722, 2′3′-c-di-AMP, or a combination of both every 5 days (shown in FIG. 16A). Tumor growth in these mice was monitored (shown in FIG. 16B). Administration of CpG-2722 and 2′3′-c-di-AMP individually exhibited some level of tumor growth suppression. Notably, the combined treatment of CpG-2722 and 2′3′-c-di-AMP demonstrated a more potent and effective suppression of tumor growth compared to the administration of either agent alone (shown in FIG. 16B). These results indicate that the combination of CpG-2722 with 2′3′-c-di-AMP exhibits a cooperative effect in regressing head and neck squamous cell carcinoma.

Those skilled in the art will readily observe that numerous modifications and alterations of the device and method may be made while retaining the teachings of the invention. Accordingly, the above disclosure should be construed as limited only by the metes and bounds of the appended claims.

What is claimed is:

1. A pharmaceutical composition, comprising:
an active pharmaceutical ingredient;
a toll-like receptor agonist;
a stimulator of interferon genes agonist; and
a pharmaceutically acceptable carrier.

2. The pharmaceutical composition of claim 1, wherein the toll-like receptor agonist is a toll-like receptor 9 agonist or a toll-like receptor 21 agonist.

3. The pharmaceutical composition of claim 2, the toll-like receptor agonist is a CpG-oligodeoxynucleotide.

4. The pharmaceutical composition of claim 3, wherein the CpG-oligodeoxynucleotide comprises a sequence being at least 70% identical to SEQ ID NO: 31.

5. The pharmaceutical composition of claim 1, wherein the stimulator of interferon genes agonist is a cyclic di-nucleotide.

6. The pharmaceutical composition of claim 5, wherein the cyclic di-nucleotide is at least one selected from the group consisting of 2′3′-cGAMP, 3′3′-cGAMP, 2′3′-cGAM(PS)2, c-di-AM(PS)2, c-di-GMP, c-di-AMP, 2′2′-cGAMP, and 2′3′-c-di-AM(PS)2.

7. A method for inducing an immune response in a subject in need thereof, comprising administering an effective amount of a pharmaceutical composition to the subject, wherein the pharmaceutical composition comprises:
an active pharmaceutical ingredient;
a toll-like receptor agonist;
a stimulator of interferon genes agonist; and
a pharmaceutically acceptable carrier.

8. The method of claim 7, wherein the toll-like receptor agonist is a toll-like receptor 9 agonist or a toll-like receptor 21 agonist.

9. The method of claim 8, the toll-like receptor agonist is a CpG-oligodeoxynucleotide.

10. The method of claim 9, wherein the CpG-oligodeoxynucleotide comprises a sequence being at least 70% identical to SEQ ID NO: 31.

11. The method of claim 7, wherein the stimulator of interferon genes agonist is a cyclic di-nucleotide.

12. The method of claim 11, wherein the cyclic di-nucleotide is at least one selected from the group consisting of 2′3′-cGAMP, 3′3′-cGAMP, 2′3′-cGAM(PS)2, c-di-AM(PS)2, c-di-GMP, c-di-AMP, 2′2′-cGAMP, and 2′3′-c-di-AM(PS)2.

13. The method of claim 7, wherein the effective amount is from 0.01 mg/kg body weight to 20 mg/kg body weight.

14. The method of claim 7, wherein the pharmaceutical composition is administered to the subject by intramuscular injection, subcutaneous administration, nasal administration, or intratumoral administration.

15. A method for treating or preventing cancer or an infectious disease, comprising administering an effective amount of a pharmaceutical composition to a subject in need thereof, wherein the pharmaceutical composition comprises:
an active pharmaceutical ingredient;
a toll-like receptor agonist;
a stimulator of interferon genes agonist; and
a pharmaceutically acceptable carrier.

16. The method of claim 15, the active pharmaceutical ingredient is an immune check point inhibitor.

17. The method of claim 16, wherein the immune check point inhibitor inhibits programmed cell death protein 1 receptor, programmed cell death ligand 1 receptor, cytotoxic T-lymphocyte-associated antigen 4 receptor, or any combination thereof.

18. The method of claim 15, wherein the toll-like receptor agonist is a CpG-oligodeoxynucleotide, and the stimulator of interferon genes agonist is a cyclic di-nucleotide.

19. The method of claim 18, wherein the CpG-oligodeoxynucleotide comprises the sequence being at least 70% identical to SEQ ID NO: 31, and the cyclic di-nucleotide is at least one selected from the group consisting of 2′3′-cGAMP, 3′3′-cGAMP, 2′3′-cGAM(PS)2, c-di-AM(PS)2, c-di-GMP, c-di-AMP, 2′2′-cGAMP, and 2′3′-c-di-AM(PS)2.

20. The method of claim 15, wherein the cancer is at least one selected from the group consisting of head and neck cancer, breast cancer, prostate cancer, melanoma, lymphoma, non-small-cell lung cancer, basal cell carcinoma, glioblastoma, and ovarian cancer, and the infectious disease is induced by hepatitis B virus, anthrax, malaria, pneumonia, herpes simplex virus, influenza virus, or any combination thereof.


SEQUENCE LISTING (31 sequences)

SEQ ID NO: 1 - DNA, 20 bp, synthetic construct
  source 1..20: mol_type = other DNA
agagcttcaggcaggcagta

SEQ ID NO: 2 - DNA, 20 bp, synthetic construct
  source 1..20: mol_type = other DNA
aggtgctcatgtcctcatcc

SEQ ID NO: 3 - DNA, 20 bp, synthetic construct
  source 1..20: mol_type = other DNA
ccggagaggagacttcacag

SEQ ID NO: 4 - DNA, 20 bp, synthetic construct
  source 1..20: mol_type = other DNA
tttccacgatttcccagaga

SEQ ID NO: 5 - DNA, 21 bp, synthetic construct
  source 1..21: mol_type = other DNA
catttgggagacctgagaaca

SEQ ID NO: 6 - DNA, 21 bp, synthetic construct
  source 1..21: mol_type = other DNA
tggagtcccgtagaaaattcc

SEQ ID NO: 7 - DNA, 20 bp, synthetic construct
  source 1..20: mol_type = other DNA
acggccagagaaaaactgaa

SEQ ID NO: 8 - DNA, 20 bp, synthetic construct
  source 1..20: mol_type = other DNA
ctaccaaggcacagggtcat

SEQ ID NO: 9 - DNA, 20 bp, synthetic construct
  source 1..20: mol_type = other DNA
cacgcctgaagaagatgaca

SEQ ID NO: 10 - DNA, 20 bp, synthetic construct
  source 1..20: mol_type = other DNA
agtccctttggtccagtgtg

SEQ ID NO: 11 - DNA, 22 bp, synthetic construct
  source 1..22: mol_type = other DNA
catgctagcctggaacgcacat

SEQ ID NO: 12 - DNA, 22 bp, synthetic construct
  source 1..22: mol_type = other DNA
actggctgttgtccttgagtcc

SEQ ID NO: 13 - DNA, 20 bp, synthetic construct
  source 1..20: mol_type = other DNA
acggcatggatctcaaagac

SEQ ID NO: 14 - DNA, 19 bp, synthetic construct
  source 1..19: mol_type = other DNA
gtgggtgaggagcacgtag

SEQ ID NO: 15 - DNA, 22 bp, synthetic construct
  source 1..22: mol_type = other DNA
atccagaaggctcaagccatcc

SEQ ID NO: 16 - DNA, 22 bp, synthetic construct
  source 1..22: mol_type = other DNA
ggagggttgtattccaagcagc

SEQ ID NO: 17 - DNA, 22 bp, synthetic construct
  source 1..22: mol_type = other DNA
gcctttgccatccaagagatgc

SEQ ID NO: 18 - DNA, 22 bp, synthetic construct
  source 1..22: mol_type = other DNA
acactgtctgctggtggagttc

SEQ ID NO: 19 - DNA, 23 bp, synthetic construct
  source 1..23: mol_type = other DNA
cagcaacagcaaggcgaaaaagg

SEQ ID NO: 20 - DNA, 22 bp, synthetic construct
  source 1..22: mol_type = other DNA
tttccgcttcctgaggctggat

SEQ ID NO: 21 - DNA, 23 bp, synthetic construct
  source 1..23: mol_type = other DNA
gcggcatgttctggatttgactc

SEQ ID NO: 22 - DNA, 22 bp, synthetic construct
  source 1..22: mol_type = other DNA
ccaccacagttgctgactcatc

SEQ ID NO: 23 - DNA, 24 bp, synthetic construct
  source 1..24: mol_type = other DNA
atcatcggcattttgaacgaggtc

SEQ ID NO: 24 - DNA, 22 bp, synthetic construct
  source 1..22: mol_type = other DNA
accttggaagccctacagacga

SEQ ID NO: 25 - DNA, 22 bp, synthetic construct
  source 1..22: mol_type = other DNA
gatgaggcttcctgtccctact

SEQ ID NO: 26 - DNA, 25 bp, synthetic construct
  source 1..25: mol_type = other DNA
tgacaggttttggaatagcatttcc

SEQ ID NO: 27 - DNA, 22 bp, synthetic construct
  source 1..22: mol_type = other DNA
aacggcagcatggtatggagtg

SEQ ID NO: 28 - DNA, 22 bp, synthetic construct
  source 1..22: mol_type = other DNA
tgggtcctgtagatggcattgc

SEQ ID NO: 29 - DNA, 23 bp, synthetic construct
  source 1..23: mol_type = other DNA
cattgctgacaggatgcagaagg

SEQ ID NO: 30 - DNA, 22 bp, synthetic construct
  source 1..22: mol_type = other DNA
tgctggaaggtggacagtgagg

SEQ ID NO: 31 - DNA, 19 bp, synthetic construct
  source 1..19: mol_type = other DNA
gttgtcgttttttgtcgtt